(19) United States
(12) Patent Application Publication
(10) Pub. No.: US 2025/0334546 A1
(43) Pub. Date: Oct. 30, 2025

(54) METHOD FOR RAPID DETECTION OF PFAS

(71) Applicant: NEW JERSEY INSTITUTE OF TECHNOLOGY, Newark, NJ (US)

(72) Inventors: Hao Chen, Livingston, NJ (US); Md. Tanim-Al Hassan, Harrison, NJ (US)

(73) Assignee: NEW JERSEY INSTITUTE OF TECHNOLOGY, Newark, NJ (US)

(21) Appl. No.: 19/189,055

(22) Filed: Apr. 24, 2025

Related U.S. Application Data
(60) Provisional application No. 63/638,065, filed on Apr. 24, 2024.

(51) Int. Cl. G01N 27/62; G01N 33/44

(52) U.S. Cl. CPC G01N 27/62; G01N 33/44

(57) ABSTRACT

Embodiments are directed to paper spray (PS)-based mass spectrometry (MS) techniques for fast and highly selective detection of PFAS. The PS-MS techniques can utilize filter paper for sample loading and ionization of samples. Solid materials containing PFAS can be directly used as samples in the PS-MS techniques. For samples containing ion suppression matrices, desalting paper spray mass spectrometry (DPS-MS) rapidly desalts, ionizes, and detects PFAS species by retaining the PFAS on the filter paper while selectively washing away the ion suppression matrices. The DPS-MS method is highly sensitive and can be applicable to directly analyze soil and sediment samples and extracts.

### CROSS-REFERENCE TO RELATED APPLICATIONS

This application claims priority to U.S. Provisional Application No. 63/638,065, filed Apr. 24, 2024, the entire disclosure of which is hereby incorporated by reference herein.

### STATEMENT REGARDING FEDERALLY SPONSORED RESEARCH

This invention was made with government support under Agreement No. CHE-2203284 awarded by the National Science Foundation. The government has certain rights in the invention.

### TECHNICAL FIELD

Embodiments of the present disclosure generally relate to methods for rapid and sensitive detection of per- and polyfluoroalkyl substances (PFAS) using paper spray-based mass spectrometry techniques. In particular, embodiments of the present disclosure provide methods that require little to no sample processing and are capable of detecting ppt levels of PFAS in a sample.

### BACKGROUND

Per- and poly-fluoroalkyl substances (PFAS), widely recognized as “forever chemicals”, are a class of organic substances in which backbone hydrogens are substituted with fluorine atoms. Perfluoroalkyl chains (CₙF₂ₙ₊₁) with polar heads (e.g., CO₂_{^{−}}, SO₃_{^{−}}) are a common structural feature of PFAS molecules (Sivagami et al, Sci. of The Total Env., 2023; 861). PFAS are man-made chemicals with unique surfactant properties and oil/water repellency and have been used in manufacturing, consumer, and industrial products since the 1960s PFAS are very stable due to the high C—F bond energy (531.5 kj·mol⁻¹), making them resistant to degradation and environmentally persistent (Barreca et al, J. of Chem., 2018).

The Organization for Economic Cooperation and Development (OECD) global database reveals that more than 4,700 PFAS-related CAS numbers have been identified with manifold physicochemical properties. Based on the recently revised definition of PFAS to include any chemical containing at least one saturated CF₂ or CF₃ moiety, PubChem, one of the largest open chemical collections, now contains over 7 million PFAS (Schymanski et al, Environ. Sci. & Technol., 2023;57 (44):16918-16928). Among commonly found PFAS species, PFOA and PFOS are of great concern within this family of compounds because of their persistence, toxicity, and potential bioaccumulation in the environment. From a regulatory point of view, PFOA and PFOS have been included by the Stockholm Convention as persistent organic pollutants in Annex A and B, suggesting their eradication and production restrictions (Stahl et al, Env. Sci Europe., 2011; 23). These compounds have been linked to several health issues, including risk of cancer and birth defects, with exposure to PFOA and/or PFOS causing adverse effects on fetus development, including decreased birth weight. Updated health advisories for PFOA and PFOS include an additional adverse effect of the suppression of vaccine response, causing a decrease in serum antibody concentrations in children.

Recent reports indicate that PFAS are present in different matrices, such as food, water, biological samples, and soil with varying levels (e.g., 1 ppt to 237 ppb) (Rankin et al., Chemosphere., 2016; 161:333-341; Ahmadireskety et al., Sci. of The Total Environ., 2021; 760:143944 and Brusseau et al., Sci Total Environ., 2020; 740:140017). The study of PFAS in food packaging materials and soils has received much attention. Packaging has become a crucial aspect of food manufacturing as it serves several vital purposes, such as safeguarding food from external factors, enabling preservation and convenient transportation, and furnishing consumers with information about ingredients and nutrition. Over recent years, the food industry has witnessed a substantial increase in both the production and use of packaging materials to satisfy the high demand. Remarkably, food packaging now constitutes nearly two-thirds of the overall volume of packaging waste. While the packaging manufacturing sector endeavors to create materials that minimize environmental impact while ensuring food safety, packaging as a potential source of food contamination is a growing concern. This is primarily due to substances migrating from the packaging into the food. Fluorochemical compounds have emerged as a significant concern in terms of food safety. Such compounds are extensively employed as coatings on food packaging to repel grease and water {Ramírez et al, Foods, 2021; 10 (7)}.

PFAS are often found in soil, which is a complex matrix with various organic and inorganic components. Due to the complexity and variety of the matrices, detection of PFAS in soil typically requires sample preparation and/or pretreatment before analysis. Solid phase extraction (SPE), solid phase microextraction (SPME), liquid-liquid extraction (LLE), and dispersive liquid-liquid microextraction (DLLME) are extraction techniques commonly reported to remove sample matrices and to preconcentrate samples prior to analysis. However, such extraction techniques result in low recovery yields of analytes and false negative results in trace analyses. Due to the demand for high sensitivity and low limit of detection (LOD) for trace analysis of PFAS, liquid chromatography (LC) coupled with electrospray ionization-tandem mass spectrometry (ESI-MS/MS) or high-resolution mass spectrometry (HRMS) is mostly used after sample pretreatment for targeted and non-targeted analyses. While LC/MS provides a foundation for PFAS analysis, it possesses several drawbacks. For example, time-consuming LC separation steps, excessive solvent consumption, and generation of large amounts of chemical waste make LC techniques incompatible with the need for high-throughput analyses, given that separation times using such techniques are in the magnitude of tens of minutes (Kurwadkar et al, Sci Total Environ., 2022; 809:151003).

Another challenge for PFAS analysis of environmental samples by MS techniques is the ion signal suppression by the matrix. Thus, the matrix needs to be removed before performing MS analysis. While matrix removal might be accomplished by extraction prior to MS analysis, such sample preparation steps take a significant amount of time. For instance, to analyze a PFAS coated packaging material such as a specially microwaved popcorn paper, Zabaleta et al., first carried out a burdensome extraction procedure (Zabaleta et al., Food Chem., 2017; 230:497-506.). The procedure required cutting the paper into a certain size (1 dm²) and soaking the paper in a vessel containing methanol. After soaking, the paper required sonication and then evaporation to dry. Finally, the sample was reconstituted for subsequent LC/MS analysis. The entire process is time consuming. Likewise, sample preparation for LC/MS analysis of PFAS in soil samples is troublesome. Not only is the process time-consuming due to the intricate extraction and cleanup processes involved, it is also labor-intensive and requires substantial manual effort. Moreover, the specialized consumables and equipment necessary for sample preparation, such as solid phase extraction (SPE) cartridges and solvents, add to the financial burden of these PFAS analysis methods.

Achieving the necessary sensitivity for PFAS detection is a further challenge, especially when dealing with complex sample matrices and/or low-level PFAS compounds. Simon et al. reported a solid phase extraction (SPE) method to extract organically bound fluorine (EOF) from soil samples, followed by quantifying the PFAS by high resolution-continuum source-graphite furnace molecular absorption spectrometry (HR-CS-GFMAS) (Simon et al., Chemosphere., 2022; 295:133922). The procedural LOD was shown to be 3.43 μg/kg (3.43 ppb). Yeung et al. performed similar studies and quantified PFOS present in the surface and core sediment near Lake Ontario using a LC/MS method, in which LOD for PFOS was found to be 30.1 μg/kg (30.1 ppb). Rankin et al. reported PFAS soil concentrations for a single sampling site located in Antarctica. After collecting the soil sample, Rankin performed extraction by mixing the soil with methanol in a centrifuge tube, then sodium hydroxide and ACN:water (90:10) was added for vortexing (15˜30 s), followed by sonicating for 60 min in an ice bath. After extraction, the sample was passed to a SPE manifold. PFOA and PFOS concentrations were measured to be 0.05 and 0.007 μg/kg (50 ppt and 7 ppt), respectively, as analyzed by LC/MS/MS. Recently, a 3D-printed cone ionization strategy was reported for in situ analysis of per- and polyfluoroalkyl substances in soils and sediments, where a 1 g sample was deposited in the cone cavity (Brown H M, Fedick P W., Chemosphere. 2021; 272:129708). PFAS was extracted and eluted by adding 1 mL of methanol to the cone for spray ionization with −5.75 kV high voltage. The method showed LOD at 100 ppt level. In that study, however, cone clogging was a challenge after sample deposition.

Paper spray mass spectrometry (PS-MS), pioneered by Cooks, Ouyang, and their colleagues, provides an analytical technique that requires minimal or no purification steps. PS-MS has gained prominence as one of the most extensively employed ambient ionization methods to analyze a diverse range of compounds, including drugs, peptides, proteins, reaction intermediates, various food components, metabolites, and environmental pollutants. However, few studies for PFAS analysis by PS-MS are reported. Sero et al. reported an analysis of neutral fluorinated compounds, particularly fluorotelomer alcohols (FTOHs), fluoroctane sulfonamides (FOSAs) and fluorooctane sulfonamido-ethanols (FOSEs), by photoionization paper spray. According to Sero, a high energy UV-krypton light beam was required to ionize the sample. The most intense ions observed in the mass spectra were [M−H]⁻ for FOSAs and [M+O₂]⁻ for FTOHs and FOSEs, respectively, and a quantitation sensitivity of mg·L⁻¹ (or ppm) was reported (Seró et al., Anal Chim Acta., 2022; 1204:339720).

Therefore, there remains an ongoing need for more rapid methods of detecting low levels of PFAS in a variety of materials. There is a further need for methods of detecting low levels of a wide variety of PFAS, including acidic PFAS compounds such as PFOA and PFOS, which are of great concern because of their persistence, toxicity, and potential bioaccumulation in the environment.

### SUMMARY

One aspect of the present disclosure pertains to a method for detecting the presence of one or more per- and polyfluorinated alkyl substances (PFAS) in a sample, the method comprising analyzing the sample using a paper spray mass spectrometry (PS-MS) technique to detect the one or more PFAS, wherein the PS-MS technique uses high voltage spray ionization.

Another aspect of the present disclosure pertains to a method for detecting the presence of one or more per- and polyfluorinated alkyl substances (PFAS) in a sample, the method comprising obtaining a sample and analyzing the sample using a paper spray mass spectrometry (PS-MS) technique to detect the one or more PFAS, wherein the sample analyzed by the PS-MS technique is either unprocessed or minimally processed. Embodiments according to this aspect can include one or more of the following features.

Another aspect of the present disclosure pertains to a method for detecting the presence of one or more per- and polyfluorinated alkyl substances (PFAS) in a sample containing one or more ion suppression matrices, the method comprising obtaining the sample and analyzing the sample using a desalting paper spray mass spectrometry (DPS-MS) technique to detect the one or more PFAS. The DPS-MS technique comprises applying the sample to a filter paper, performing a desalting step to remove the ion suppression matrices from the filter paper, positioning the filter paper in front of a mass spectrometry inlet, eluting the one or more PFAS by wetting the filter paper with an elution solvent, and applying a voltage to the wetted filter paper to ionize the one or more PFAS.

Embodiments according to the above aspects of the present disclosure can include one or more of the following features.

In one or more embodiments, the PS-MS technique comprises applying the sample to a filter paper, positioning the filter paper in front of a mass spectrometry inlet, eluting the one or more PFAS by wetting the filter paper with an elution solvent, and applying a voltage to the wetted paper to ionize the one or more PFAS.

In one or more embodiments, the method provides detection of the one or more PFAS within a limit of detection of about 0.01 ppt to about 100 ppt.

In one or more embodiments, the method is completed within about 6 minutes, within about 5 minutes, within about 4 minutes, within about 3 minutes, within about 2 minutes, within about 1 minute, within about 30 seconds, within about 20 seconds, or within about 10 seconds.

In one or more embodiments, the sample is selected from water, soil, air, plants, food, sludge, vegetables, meats, packaging materials, plastic products, toys, cosmetics, agricultural products, pharmaceutical products, electronics, consumer products, and solvent extracts thereof.

In one or more embodiments, the sample is an unprocessed sample.

In one or more embodiments, prior to analyzing the sample, the method further comprises extraction of the sample with a solvent, wherein analyzing the sample comprises analyzing the sample extract, and wherein the method is completed within about 6 minutes, within about 5 minutes, within about 4 minutes, within about 3 minutes, within about 2 minutes, or within about 1 minute.

In one or more embodiments, the sample is a solid material containing one or more PFAS, analyzing the sample using a PS-MS comprises directly analyzing the solid material, and applying the PS-MS technique comprises cutting the solid material containing one or more PFAS into a cut sample shape, positioning the cut sample shape in front of a mass spectrometry inlet, eluting the one or more PFAS by wetting the cut sample shape with an elution solvent, and applying a voltage to the wetted cut sample shape to ionize the one or more PFAS. In one or more embodiments, the solid material is a food packaging material, a plant, a leaf, a vegetable, a fruit, a paper material, or a plastic material.

In one or more embodiments, about 1 μL to about 100 μL of sample is applied to the filter paper or the cut sample shape at least one time and up to four times. In one or more embodiments, positioning the filter paper or cut sample shape in front of the mass spectrometry inlet comprises positioning the filter paper or cut sample shape about 2 mm to about 50 mm in front of the mass spectrometry inlet. In one or more embodiments, the voltage ranges from about 1 kV to about 10 kV. In one or more embodiments, the elution solvent is an organic solvent, water, or a combination of organic solvent and water, or a solvent containing a derivatizing reagent. Suitable a derivatizing reagents can be suitably selected and, for example, can include but are not limited to, 2-fluoro-N-methylpyridinium p-toluenesulfonate.

In one or more embodiments, a desalting step is performed, the desalting step comprising, after applying the prepared sample to the filter paper, adding water to the filter paper to wick away the ion suppression matrices.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1A schematically illustrates the apparatus and workflow of paper spray mass spectrometry (PS-MS) according to embodiments of the present disclosure, and FIG. 1B schematically illustrates the apparatus and workflow of desalting paper spray mass spectrometry (DP S-MS) according to embodiments of the present disclosure.

FIG. 2A-E graphically illustrate the negative ion mode PS-MS spectra of a variety of food packaging materials using PS-MS according to embodiments of the present disclosure, where the food packaging materials analyzed were: Microwave popcorn paper (FIG. 2A), Burger King French fry box (FIG. 2B), Instant noodles box (Beef Pro) (FIG. 2C), McDonald's burger wrapper (FIG. 2D), and Burger King wrapper (FIG. 2E). As illustrated, several PFAS including PFBA, PFPeA, PFHxA, 5:3 FTCA, 5:3 FTUCA, 6:2 FTUCA, PFHpA, 6:2 FTCA, PFOA, PFNA and PFOS were found in these food packaging materials.

FIG. 3 is a table providing PFAS identified from the food packaging materials in FIGS. 2A-E, with their chemical formulas, precursor ions, and fragmented ions;

FIG. 4 illustrates SEM images of Whatman p8 filter paper a) before and b) after loading PFOS; and EDX images of Whatman p8 filter paper c) before and d) after loading PFOS;

FIG. 5 graphically illustrates the negative ion mode PS-MS spectrum using PS-MS according to embodiments of the present disclosure for a triangular-shaped blank paper with no sample loaded;

FIG. 6 graphically illustrates the negative ion mode extracted ion chromatograms (EIC) spectra of PFAS ions detected from microwave popcorn paper (Panels a-k), Burger King French fry box (Panels l-m), Instant Noddles box (Panels n-p), McDonald's burger wrapper (Panel q), and Burger King wrapper (Panel r);

FIGS. 7A-I graphically illustrate the negative ion mode PS-MS spectra of PFAS ions from standard PFAS samples: a) PFBA, b) PFPeA, c) PFBS, d) PFHxA, e) PFHpA, f) 6:2 FTCA, g) PFOA, h) PFNA and i) PFOS, using PS-MS according to embodiments of the present disclosure;

FIGS. 8A-F graphically illustrates DPS-MS parameter optimization by measuring a PFOA signal using DPS-MS according to embodiments of the present disclosure while varying a) distance between the filter paper and MS inlet, b) sample loading volume, c) spray solvent (elution solvent) volume, d) spray ionization voltage, e) elution solvent, and f) times/cycles of desalting;

FIG. 9 graphically illustrates the extracted ion chromatogram (EIC) for the ion of m/z 412.9 from 50 μM PFOA in 50 mM KCl acquired using DPS-MS according to embodiments of the present disclosure;

FIG. 10 graphically illustrates results of a DPS-MS recovery test for a PFOA ion signal from a) a standard PFOA without 50 mM KCl and b) PFOA with 50 mM KCl (triplicate measurements) using DPS-MS according to embodiments of the present disclosure;

FIG. 11 graphically illustrates negative ion mode MS spectra of 50 μM PFOA in 50 mM KCl collected using a) nanoESI-MS (no MeOH was added), b) nanoESI-MS (MeOH was added), and c) DPS-MS; and d) collision-induced dissociation (CID) MS/MS spectrum of the PFOA ion C₇F₁₅COO⁻ at m/z 412.9;

FIG. 12 graphically illustrates negative ion mode EIC spectra of [PFOA-H]⁻ (m/z 412.9) detected a) from 50 μM PFOA in 50 mM KCl (water as the sample solvent) by a conventional nanoESI-MS, b) from 50 μM PFOA in 50 mM KCl (water/methanol as the sample solvent) by a conventional nanoESI-MS, and c) from 50 μM PFOA in 50 mM KCl (water as the sample solvent) by DPS-MS according to embodiments of the present disclosure;

FIG. 13 graphically illustrates negative ion mode nanoESI-MS spectra of 1 nM PFOA in a) 50 mM NaCl and c) 50 mM Na₂SO₄; and negative ion mode DPS-MS spectra obtained using DPS-MS according to embodiments of the present disclosure for 1 nM PFOA in b) 50 mM NaCl and d) 50 mM Na₂SO₄;

FIG. 14 graphically illustrates negative ion mode DPS-MS spectra obtained using DPS-MS according to embodiments of the present disclosure for a) 50 μM PFOS in 50 mM KCl, b) 50 μM PFHxA in 50 mM KCl, and c) 50 μM PFBS in 50 mM KCl. PFOS: measured m/z 498.93062, theoretical m/z 498.93022 and mass error: 0.8 ppm; PFHxA: measured m/z 312.97290, theoretical m/z 312.97281 and mass error: 0.3 ppm; PFBS: measured m/z 298.9429, theoretical m/z 298.94299 and mass error: −0.3 ppm;

FIG. 15 graphically illustrates CID MS/MS spectra of a) deprotonated PFOS at m/z 498.9, b) deprotonated PFHxA at m/z 312.9, and c) deprotonated PFBS at m/z 298.9;

FIG. 16 graphically illustrates negative ion mode DPS-MS spectra obtained using DPS-MS according to embodiments of the present disclosure for 100 pM of a) PFOA, b) PFOS, c) PFHxA, and d) PFBS in 50 mM KCl;

FIG. 17 graphically illustrates negative ion mode DPS-MS spectra obtained using DPS-MS according to embodiments of the present disclosure for a) blank paper with 50 mM KCl, and b) 10 pM of PFOA in 50 mM KCl;

FIG. 18 graphically illustrates negative ion mode MS spectra of a water extract of soil acquired by a) PS-MS without water desalting according to embodiments of the present disclosure, and b) DPS-MS with water desalting according to embodiments of the present disclosure;

FIG. 19 graphically illustrates CID MS/MS spectra of a) deprotonated PFHxA at m/z 312.9 and b) deprotonated PFBS at m/z 298.9, resulting from DPS-MS analysis of a soil extract according to embodiments of the present disclosure;

FIG. 20 graphically illustrates the negative ion mode DPS-MS spectrum of a soil extract using DPS-MS according to embodiments of the present disclosure;

FIG. 21 graphically illustrates the negative ion mode MS spectra of an unprocessed soil sample acquired using a) PS-MS according to embodiments of the present disclosure (without water desalting) and b) DPS-MS according to embodiments of the present disclosure (with water desalting);

FIG. 22 graphically illustrates the negative ion mode PS-MS spectra using PS-MS according to embodiments of the present disclosure for a) blank paper with no sample loaded, b) paper loaded with New Jersey Institute of Technology (NJIT) lab tap water, c) paper loaded with tap water from Harrison city, and d) paper loaded with NJIT fountain water;

FIG. 23 graphically illustrates the negative ion mode PS-MS spectra of NJIT lab tap water sample spiked with PFBS, PFHxA and PFOS (100 ppt each), showing the detection of: PFBS by LC/MS (FIG. 23A), PFBS by PS-MS according to embodiments of the present disclosure (FIG. 10B), PFHxA by LC/MS (FIG. 23C), PFHxA by PS-MS according to embodiments of the present disclosure (FIG. 23D), PFOA by LC/MS (FIG. 23E), PFOA by PS-MS according to embodiments of the present disclosure (FIG. 23F), and PFOS by LC/MS (FIG. 23G), and PFOS by PS-MS according to embodiments of the present disclosure (FIG. 23H);

FIG. 24 graphically illustrates the PS-MS spectrum using PS-MS according to embodiments of the present disclosure for wastewater collected from an anonymous location;

FIG. 25 graphically illustrates the negative ion mode PS-MS spectra using PS-MS according to embodiments of the present disclosure for a) Kiss Lengthen & Define Washable & Waterproof Mascara, b) Ruby Kisses Brush Concealer & Foundation, and c) Ruby Kisses Tattoo Felt Tip Eyeliner;

FIG. 26 schematically illustrates an apparatus and workflow of PS-MS according to embodiments of the present disclosure, in which the PS-MS method is further adapted for trace P FAS analysis from a water sample;

FIG. 27 graphically illustrates the negative ion mode PS-MS spectra of a) water with no PFOA and b) water with 0.1 ppt PFOA using p8 filter paper in a PS-MS apparatus and workflow illustrated in FIG. 26 according to embodiments of the present disclosure;

FIG. 28 graphically illustrates the negative ion mode PS-MS spectra of a) water with no PFOA and b) water with 0.1 ppt PFOA using membrane filter paper in the PS-MS apparatus and workflow illustrated in FIG. 26 according to embodiments of the present disclosure;

FIG. 29 schematically illustrates an apparatus and workflow of a PS-MS technique for analyzing an air sample according to embodiments of the present disclosure;

FIG. 30 graphically illustrates the negative ion mode EIC spectra of a) PFHxA ion obtained from direct analysis of soil by PS-MS according to embodiments of the present disclosure, b) PFHxA ion obtained from direct analysis of soil by DPS-MS according to embodiments of the present disclosures, and c) PFBS ion obtained from direct analysis of soil by DPS-MS according to embodiments of the present disclosure;

FIG. 31 is a table providing the raw data values of PFBS analysis with I.S. (400 pM PFOA) in triplicate measurements;

FIG. 32 illustrates a calibration curve of PFBS using the raw data in FIG. 31;

FIG. 33 is a table providing the raw data values of PFHxA analysis with I.S. (400 pM PFOA) in triplicate measurements;

FIG. 34 illustrates a calibration curve of PFHxA using the raw data in FIG. 33;

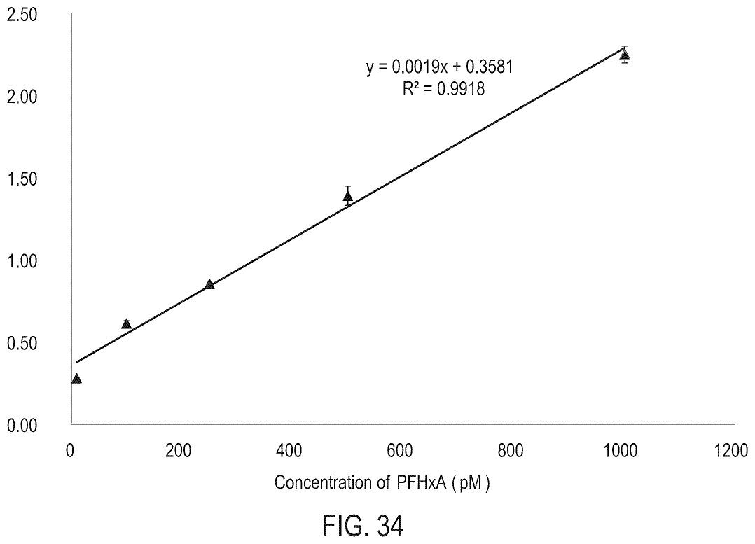

FIG. 35 is a table providing the raw data values of PFOA analysis with I.S. (400 pM PFBS) in triplicate measurements;

FIG. 36 illustrates a calibration curve of PFOA using the raw data in FIG. 35;

FIG. 37 is a table providing raw data values of PFOS analysis with I.S. (400 pM PFOA) in triplicate measurements; and

FIG. 38 illustrates a calibration curve of PFOS using the raw data in FIG. 37; and

FIG. 39 is a table providing observed fragment ions from PFAS ions from standard PFAS samples upon collision-induced dissociation (CID).

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

As used in this specification and the appended claims, the term “unprocessed sample” when referencing a particular sample (e.g., a water sample, soil sample, food packaging material sample, air sample, plastic product sample, cosmetics sample, etc.) refers to a sample collected from a site and used as a sample without subjecting the collected sample to any processing steps that change the properties of the sample (e.g., extraction, dissolution of the sample in water or any other solvent—where suitable other solvents can include, for example, methanol, acetonitrile, dichloromethane, chloroform, isopropanol, and the like). It is understood that changing the size or shape of a sample by, for example, cutting or tearing or otherwise manipulating a packaging material, food wrapper, or the like into a certain shape and/or size, is not a processing step that changes the properties of the sample and, thus, a cut piece of a packaging material, food wrapper, or similar material is considered to be included in the definition of an “unprocessed sample”. One example of a processing method that would result in a sample being considered “processed” is solid phase extraction (SPE).

As used in this specification and the appended claims, the term “minimal” when referring to “sample preparation or processing” refers to, after a sample is collected from a site and before it is used as a sample in PS-MS/DPS-MS, any amount or type of preparation or processing of the sample that does not increase the total time allowed for a rapid PS-MS/DPS-MS technique above an upper threshold according to the embodiments described herein. In particular, according to some embodiments described herein, a rapid PS-MS/DPS-MS technique is one in which the time between obtaining the sample and detecting the one or more PFAS is less than about 6 minutes, and in some embodiments is less than about 3 minutes, or even less than about 1 minute. The preparation and processing of the sample would occur after obtaining the sample and, thus, to be considered “minimal” the amount of time to prepare and process the sample could not increase the total time (as measured from obtaining the sample to detecting the one or more PFAS) above the upper threshold. Suitable upper thresholds of total time (as measured from obtaining the sample to detecting the one or more PFAS) are further described herein, with 6 minutes being an upper threshold of total time according to some embodiments.

As used in this specification and the appended claims, the terms “direct” and “directly” when used in connection with a sample (e.g., where a sample is “directly analyzed”, “directly ionized”, “directly infused”, “directly spotted”, “directly applied”, etc.) refers to a process in which a sample (e.g., a water sample, soil sample, food packaging material, or the like) is obtained from a site or location and, in its unprocessed state, is analyzed, ionized, infused, spotted, applied, etc. using the PS-MS techniques of the present disclosure.

As used in this specification and the appended claims, the term “high voltage” when used to describe the parameters of the PS-MS and DPS-MS methods, and specifically to refer to the spray ionization conditions, refers to a voltage of at least about −3 kV. According to embodiments described herein, this “high voltage” is a negative voltage. This “high voltage” spray ionization is understood to exclude photoionization.

As used in this specification and the appended claims, the term “limit of detection (LOD)” refers to the lowest concentration or quantity of a substance being measured that can be reliably detected with a given analytical method, where the signal of sample at the LOD level would be reliably distinguished from background noise.

The embodiments described herein generally provide improved paper spray mass spectrometry (PS-MS) and desalting paper spray mass spectrometry (DPS-MS) methods for detecting PFAS in a variety of substances. Embodiments of the PS-MS and DPS-MS techniques provide increased speed, making it possible to analyze a sample with minimal or no sample preparation or processing. Embodiments of the PS-MS and DPS-MS techniques further provide increased sensitivity, making it possible to detect PFAS in a sample at levels on the order of ppt (parts per trillion).

It is noted that throughout this specification, when referring generally to paper spray mass spectrometry (PS-MS), it is understood that desalting paper spray mass spectrometry (DPS-MS) is also encompassed. In particular, DPS-MS is an extended version of PS-MS, in which an additional step of desalting to remove a sample matrix salt is included in the general PS-MS method. Thus, when describing features, conditions, specifications, and capabilities of PS-MS throughout this specification, it is understood that those descriptions of features, conditions, and capabilities also encompass DPS-MS. Further, while exemplary embodiments are directed to methods for detecting PFAS, it should be understood that embodiments described herein could also be applied to detect other substances.

According to embodiments described herein, the PS-MS methods can be used to detect PFAS in any material in which PFAS can be found. In some embodiments, the types of materials that can be analyzed to detect PFAS using the PS-MS methods described herein include, but are not limited to: water (e.g., drinking water, ground water, waste water, etc.), soil, food, air, sludge, vegetables, meats, plants, and personal and household items including, but not limited to, packaging materials (e.g., general consumer product packaging, food packaging, etc.), plastic products, water-resistant fabrics, toys, consumer products, and personal care products (e.g., pharmaceutical products, electronics, cosmetics, skin care, hair care, and oral care products). According to embodiments described herein, the PS-MS methods are capable of analyzing the material with minimal or no preparation or processing.

In some embodiments, the methods for detecting the presence of one or more per- and polyfluorinated alkyl substances (PFAS) in a sample comprise obtaining the sample and analyzing the sample using a paper spray mass spectrometry (PS-MS) technique to detect the one or more PFAS, wherein the PS-MS technique is much more rapid than conventional techniques. According to some embodiments, the PS-MS techniques provide a time between obtaining the sample and detecting the one or more PFAS that is less than about 3 minutes. According to some embodiments, the time between obtaining the sample and detecting the one or more PFAS is less than about 6 min, less than about 5.5 min, less than about 5 min, less than about 4.5 min, less than about 4 min, less than about 3.5 min, less than about 3 min, less than about 2.9 min, less than about 2.8 min, less than about 2.8 min, less than about 2.7 min, less than about 2.6 min, less than about 2.5 min, less than about 2.4 min, less than about 2.3 min, less than about 2.2 min, less than about 2.1 min, less than about 2 min, less than about 1.9 min, less than about 1.8 min, less than about 1.7 min, less than about 1.6 min, less than about 1.5 min, less than about 1.4 min, less than about 1.3 min, less than about 1.2 min, less than about 1.1 min, or less than about 1 min. According to some embodiments, the time between obtaining the sample detecting the one or more PFAS ranges from about 1 min to about 3 min, from about 1 min to about 2.5 min, from about 1 min to about 2 min, or from about 1 min to about 1.5 min. According to some embodiments, the time between obtaining the sample and detecting the one or more PFAS can be as low as about 10 seconds and even as low as about 5 seconds, and in some embodiments can range from about 5 seconds to about 3 min, or from about 5 seconds to about 1 min.

In some embodiments, the methods for detecting the presence of one or more per- and polyfluorinated alkyl substances (PFAS) in a sample comprise obtaining the sample and analyzing the sample using a paper spray mass spectrometry (PS-MS) technique to detect the one or more PFAS, wherein the PS-MS technique provides increased detection sensitivity. While currently available detection techniques are capable of only detecting high levels of PFAS in samples, the present methods are capable of detecting PFAS present at a ppt limit of detection (LOD). According to some embodiments, the present PS-MS techniques provide detection of one or more PFAS at a limit of detection (LOD) as low as about 0.1 ppt, as low as about 0.5 ppt, and in some embodiments as low as about 0.01 ppt. According to some embodiments, the PS-MS techniques provide detection of the one or more PFAS within a limit of detection (LOD) of less than about 100 ppt, less than about 95 ppt, less than about 90 ppt, less than about 85 ppt, less than about 80 ppt, less than about 75 ppt, less than about 70 ppt, less than about 65 ppt, less than about 60 ppt, less than about 55 ppt, less than about 50 ppt, less than about 45 ppt, less than about 40 ppt, less than about 35 ppt, less than about 30 ppt, less than about 25 ppt, less than about 20 ppt, less than about 15 ppt, or less than about 10 ppt. As such, the present PS-MS techniques provide for trace analysis of PFAS within a sample.

In some embodiments, PS-MS technique comprises applying the obtained sample to a filter paper, positioning the filter paper in front of a mass spectrometry inlet, eluting the one or more PFAS by wetting the filter paper with an elution solvent, and applying a voltage to the wetted paper to ionize one or more PFAS. According to some embodiments, the sample applied to the filter paper is an unprocessed sample. In some embodiments, the sample applied to the paper is first subjected to minimal sample preparation and/or processing. According to some embodiments, the minimal sample preparation and/or processing comprises extracting the obtained sample with a solvent to prepare a sample extract, and the sample extract is applied to the filter paper for analysis.

In some embodiments, to examine PFAS contamination of a sample having the form of a solid material, the PS-MS method can be directly applied to the sample. For example, in the case of solid materials that maintain their structural integrity when wetted with an elution solvent, processing and application of the solid materials to a filter paper is unnecessary. Accordingly, in some embodiments, the methods for detecting the presence of one or more per- and polyfluorinated alkyl substances (PFAS) in such solid materials comprises positioning the sample in front of a mass spectrometry inlet, eluting the one or more PFAS by wetting the sample with an elution solvent, and applying a voltage to the wetted sample to ionize the one or more PFAS. According to some embodiments, the solid material is provided in a particular sample size and/or shape by cutting, tearing, or otherwise manipulating the solid material into a suitable sample size and/or shape, the sample is then positioned in front of the mass spectrometry inlet, the one or more PFAS are eluted by wetting the sample with an elution solvent, and a voltage is applied to the wetted sample to ionize the one or more PFAS. In some embodiments, the sample include, but is not limited to, food packaging materials, plants, leaves, vegetables, fruit, and various paper and plastic products.

In some embodiments, in the PS-MS method, about 1 μL to about 100 μL of sample is applied to the filter paper at least one time and up to four times. In some embodiments, about 2 μL, about 3 μL, about 4 μL, about 5 μL, about 6 μL, about 7 μL, about 8 μL, about 9 μL, about 10 μL, about 11 μL, about 12 μL, about 13 μL, about 14 μL, about 15 μL, about 16 μL, about 17 μL, about 18 μL, about 19 μL, about 20 μL, about 21 μL, about 22 μL, about 23 μL, about 24 μL, about 25 μL, about 26 μL, about 27 μL, about 28 μL, about 29 μL, about 30 μL, about 31 μL, about 32 μL, about 33 μL, about 34 μL, about 35 μL, about 36 μL, about 37 μL, about 38 μL, about 39 μL, or about 40 μL of sample is applied to the filter paper at least one time and up to four times. In some embodiments, up to about 90 μL, up to about 80 μL, up to about 70 μL, up to about 60 μL, up to about 50 μL, or up to about 40 μL of sample is applied to the filter paper at least one time and up to four times. According to some embodiments, about 40 μL of sample is applied to the filter paper. According to some embodiments, about 10 μL of sample is applied to the filter paper at least one time and up to four times, and in some embodiments about 10 μL of sample is applied to the filter paper four times.

In some embodiments, in the PS-MS method, the filter paper or the solid sample is positioned about 2 mm to about 50 mm in front of the mass spectrometry inlet. In some embodiments, the filter paper or the solid sample is positioned about 3 mm to about 40 mm, about 4 mm to about 30 mm, about 5 mm to about 20 mm, or about 6 mm to about 10 mm in front of the mass spectrometry inlet. According to some embodiments, the filter paper or the cut sample shape is positioned about 8 mm in front of the mass spectrometry inlet.

In some embodiments, in the PS-MS method, the voltage applied to the wetted filter paper or the wetted cut sample shape to ionize the one or more PFAS ranges from about −1 kV to about −10 kV. In some embodiments, the voltage is about −2kV to about −8 kV, about −2.5 kV to about −6 kV, about −3 kV to about −5 kV, about −3.5 kV to about −4 kV. According to some embodiments, the voltage is about −3.5 kV.

In some embodiments, in the PS-MS method, the elution solvent applied to the filter paper or the solid sample is an organic solvent, water, or a combination of organic solvent and water. In some embodiments, the organic solvent is methanol, acetonitrile, acetone, isopropanol, dichloromethane, or chloroform. In some embodiments, the one or more PFAS are eluted by wetting the filter paper or the solid sample with about 5 μL to about 50 μL elution solvent, about 10 μL to about 45 μL elution solvent, about 15 μL to about 40 μL elution solvent, about 20 μL to about 35 μL elution solvent, or about 25 μL to about 30 μL elution solvent.

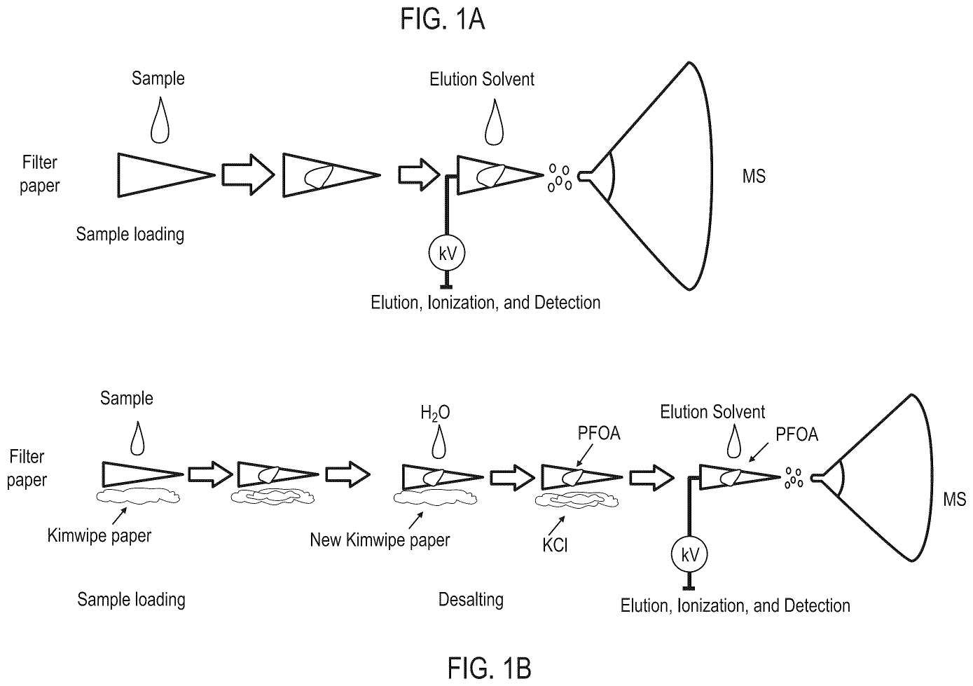

A general schematic illustrating a PS-MS technique according to an embodiment is provided in FIG. 1A, in which a sample (e.g., 10 μL-40 μL sample) is applied to a triangular filter paper, the filter paper is placed in front of a mass spectrometry inlet, the one or more PFAS are eluted by wetting the filter paper with an elution solvent (e.g., 30 μL MeOH), and a voltage is applied to the wetted paper to ionize the one or more PFAS. In some embodiments, the filter paper is Fisher brand qualitative p8 filter paper cut into a triangular shape (e.g., 10 mm×5 mm, height×width). However, any other conventional filter paper or modified filter paper in any shape and size can be suitably used. According to some embodiments, the filter paper is modified (e.g., the filter paper can contain activated carbon, hydrogels, and ion-exchange resins) to favor trapping and ionizing the one or more PFAS. As described herein, any suitable amount of the sample is applied to the filter paper once or multiple times. According to some embodiments, a 10 ∥L sample solution is applied to a cleaned filter paper, and in some embodiments the 10 ∥L of sample is applied to cleaned filter paper four times to enhance PFAS detection sensitivity. In some embodiments the sample is an unprocessed sample, while in other embodiments the sample is a minimally processed sample. For example, according to some embodiments, prior to applying the sample to the filter paper, the filter paper is cleaned by sonicating with a solution. In an exemplary embodiment, a filter paper is sonicated sequentially by acetone, methanol, and methanol/water (50:50 v/v, 15 min each). In some embodiments, the sample solution is mainly aqueous. After drying, the paper triangle is then held in front of the MS inlet (e.g., 8 mm away from the MS inlet using a high-voltage cable with an alligator clip), and a suitable elution solvent (e.g., 30 μL of MeOH (100%)) is added directly onto the filter paper to elute target compounds for ionization upon application of a high voltage (e.g., −3.5 kV) to the wetted paper.

In some embodiments, methods for detecting the presence of one or more per- and polyfluorinated alkyl substances (P FAS) in a sample containing one or more ion suppression matrices is provided. For PFAS samples containing complicated matrices (e.g., salts, soils) which cause or contribute to ion suppression (i.e., signal suppression during MS analysis), enhanced detection sensitivity is achieved by desalting paper spray mass spectrometry (DPS-MS) methods described herein. In particular, it was demonstrated that the desalting process helps to remove at least some of the sample matrix inherently present in the sample. According to embodiments described herein, it was demonstrated that after desalting, the signal in DPS-MS is improved significantly in comparison to a PS-MS method without desalting. For example, according to some embodiments, one or more PFAS in a sample that are not detectable by the present PS-MS methods are detectable when using the present DPS-MS methods. According to some embodiments, the present DPS-MS methods increase detection sensitivity by about 50% to about 200% as compared to the present PS-MS methods.

In some embodiments, methods for detecting the presence of one or more per- and polyfluorinated alkyl substances (P FAS) in a sample containing one or more ion suppression matrices comprise obtaining the sample and analyzing the sample using a desalting paper spray mass spectrometry (DPS-MS) technique to detect the one or more PFAS. According to embodiments described herein, the DPS-MS technique comprises applying the sample to a filter paper, performing a desalting step to remove at least a portion of the ion suppression matrices from the filter paper, positioning the filter paper in front of a mass spectrometry inlet, eluting the one or more PFAS by wetting the filter paper with an elution solvent, and applying a voltage to the wetted filter paper to ionize the one or more PFAS. According to some embodiments, the DPS-MS method integrates the desalting step with the ionization step on the same filter paper. It was found that application of a sample containing PFAS which contain polar heads of COO⁻ or SO₃ to a filter paper fabricated of hydrophilic cellulose provides strong interactions between the PFAS molecules and the filter paper. According to embodiments described herein, a sample is deposited onto the filter paper, and a desalting material (e.g., water) is applied to rinse away the non-volatile salt matrix while keeping PFAS retained on the paper due to these strong interactions. This allows for the analysis of PFAS in different salt matrices as well as the rapid PFAS detection from samples containing ion suppression matrices (e.g., soil and sediment samples and extracts) by DPS-MS.

In some embodiments, desalting to remove at least a portion of the ion suppression matrices would include removal of up to about 100% of the ion suppression matrices. According to some embodiments, about 50% to about 100% of the ion suppression matrices are removed in the desalting step. According to some embodiments, about 55% to about 100%, about 60% to about 100%, about 65% to about 100%, about 70% to about 100%, about 75% to about 100%, about 80% to about 100%, about 85% to about 100%, about 90% to about 100%, or about 95% to about 100% of the ion suppression matrices are removed in the desalting step. In general, removal of the ion suppression matrices is sample dependent, with some types of samples being more readily desalted than other types of samples. In some embodiments, desalting comprises adding water to the filter paper to wick away the ion suppression matrices.

According to embodiments described herein, all of the features, conditions, specifications, and capabilities described with respect to the general PS-MS method apply to the DPS-MS method including, for example, the time between obtaining the sample and detecting the one or more PFAS, the limit of detection (LOD) of the one or more PFAS, the distance between the sample and the mass spectrometry inlet, the voltage applied to the wetted filter paper, the type and amount of elution solvent applied to the filter paper, etc.

A general schematic illustrating a DPS-MS technique according to an embodiment is provided in FIG. 1B. As illustrated, a sample (e.g., 10 μL-40 μL sample) is applied to a triangular filter paper. According to some embodiments, the filter paper is placed on top of a Kimwipe to facilitate the absorption by capillarity. A desalting process is then carried out by loading a suitable desalting solvent onto the filter paper to wick the sample salts and other matrix chemicals in the sample. According to some embodiments, prior to desalting, the filter paper with the applied sample is first placed on top of a new Kimwipe to facilitate absorption of the desalting material. In some embodiments, desalting is achieved by loading about 30 μL of ultrapure H₂O (in some embodiments 10 μL of H₂O is applied three times) onto the filter paper. It will be understood that any suitable number of loadings and amount of desalting material per loading can be applied to the DPS-MS methods described herein. The paper triangle is held in front of the MS inlet (e.g. 8 mm away from the MS inlet using a high-voltage cable with an alligator clip), and a suitable elution solvent (e.g., 30 μL of MeOH (100%)) is added directly onto the filter paper to elute target compounds for ionization upon application of a high voltage (e.g., −3.5 kV) to the wetted paper. In some embodiments, the filter paper is Fisher brand qualitative p8 filter paper cut into a triangular shape (e.g., 10 mm×5 mm, height×width). However, any other conventional filter paper or modified paper in any shape and size can be suitably used. As described herein, any suitable amount of the sample is applied to the filter paper once or multiple times. According to some embodiments, a 10 μL sample solution is applied to a cleaned filter paper, and in some embodiments the 10 μL of sample is applied to the cleaned filter paper four times to enhance the detection sensitivity. In some embodiments the sample is an unprocessed sample, while in other embodiments the sample is a minimally processed sample. For example, according to some embodiments, prior to applying the sample to the filter paper, the sample is sonicated with a solvent for quick extraction. In some embodiments, the sample solution is mainly aqueous.

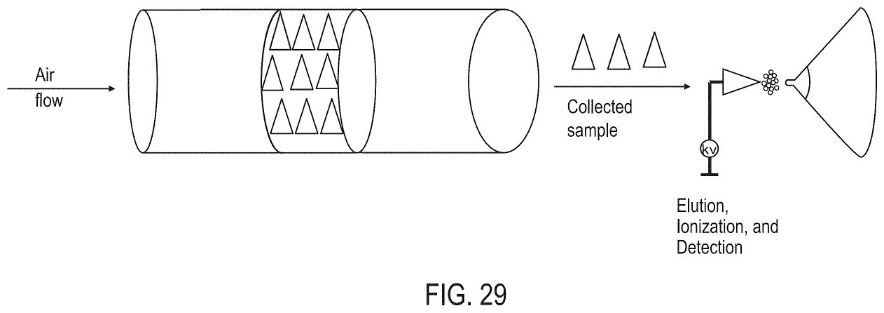

According to the embodiments described herein, the PS-MS methods are further applicable for detecting and identifying PFAS present in air. According to some embodiments, an air sample is flowed through a tube which contains filter paper, for example in the form of one or more layers of filter paper disposed in the path of the air flow. According to some embodiments, the layer of filter paper can be in the form of a plurality filter papers, for example a plurality of triangular shaped paper filters. As the air sample is flowed through the tube, the PFAS in the air sample are adsorbed onto the filter paper. The filter paper is then collected and used for the rapid detection of adsorbed PFAS by the PS-MS methods described herein. A general schematic illustrating a PS-MS technique for analyzing an air sample according to an embodiment is provided in FIG. 29.

According to embodiments described herein, the PS-MS methods are further adapted for enhanced PFAS detection sensitivity for water samples. According to some embodiments, water samples are first filtered through a filter paper. Since PFAS are adsorbed onto the filter paper during filtration, after the water sample is filtered through the filter paper, the filter paper is cut into triangular (or any other shaped) sample emitters for use in the present PS/MS methods. Any volume of water sample can be passed through the filter paper and, in some embodiments, the volume of water passed through the filter is at least 50 mL, at least 100 mL, at least 150 mL, at least 200 mL, at least 250 ml, at least 300 mL, at least 350 mL, at least 400 mL, at least 450 mL, or at least 500 mL.

The present disclosure advantageously provides methods capable of detecting PFAS in any type of sample known to contain PFAS with high sensitivity, high specificity, and fast analysis speed. Such methods are particularly beneficial because PFAS occur in a variety of materials at trace levels and, further, in complicated matrices. The disclosed paper spray-based mass spectrometry approach is both highly sensitive and rapid, requiring minimum to no sample preparation or processing. Further, high resolution mass spectrometry measurements provide accurate mass for PFAS identification with high specificity. In addition, desalting paper spray-based mass spectrometry is used for sample containing one or more ion suppression matrices (e.g., soil and sediment samples with salt matrices). The disclosed desalting techniques, likewise, are both highly sensitive and rapid, requiring minimum to no sample preparation or processing.

### EXAMPLES

### Materials, Equipment, and Methods

The materials and the methods of the present disclosure used in the Examples will be described below. While the use of specific compounds and materials is described, it is understood that the present disclosure could employ other suitable compounds and materials. Further, while specific quantities and measurements were employed, it is understood that other suitable quantities and measurements may be substituted without altering the methods embodied below.

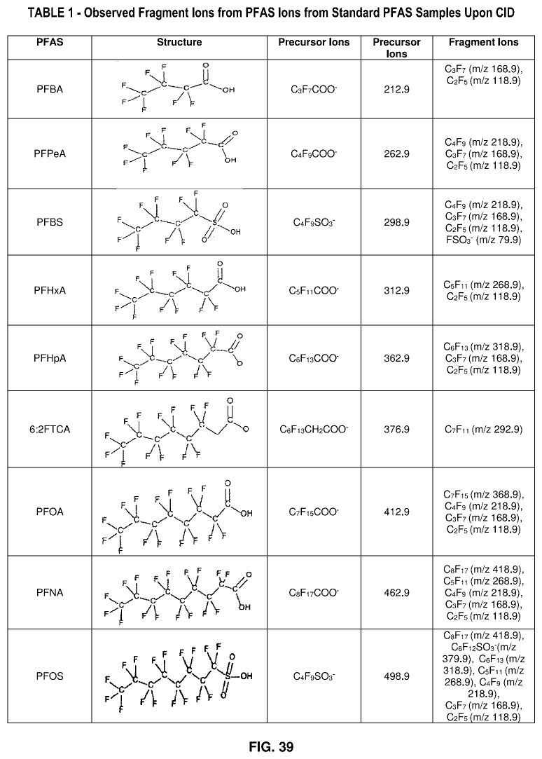

Per-fluorobutanoic acid (PFBA, CAS No. 375-22-4), per-fluoropentanoic acid (PFPeA, CAS No. 2706-90-3), per-fluoroheptanoic acid (PFHpA, CAS No. 375-85-9), per-fluorononoaic acid (PFNA, CAS No. 375-95-1), PFOA (CAS No 335-67-1), PFOS (CAS No. 1763-23-1), per-fluorohexanoic acid (PFHxA, CAS No. 307-24-4), and per-fluorobutanesulfonic acid (PFBS, CAS No. 375-73-5) were purchased from Sigma-Aldrich (St. Louis, MO, USA; Structures are shown in Table 1, FIG. 39). 2H, 2H-Perfluorooctanoic acid (6:2 FTCA, CAS No. 53826-12-3) was purchased from Synquest Laboratories (Alachua, FL). Potassium chloride, sodium chloride, sodium sulfate, HPLC-grade acetonitrile, and methanol were obtained from Fisher Scientific (Waltham, MA, United States). Deionized water was obtained from EMD Millipore (Burlington, MA, United States). A Fisher brand p8 filter paper was purchased from Fisher Scientific Co. and used in the PS-MS and DPS-MS experiments. Stock solutions of 1 mM PFOA, PFOS, PFHxA and PFBS were prepared by dissolving solid compounds in a solvent of methanol:water (20:80). Working samples were prepared by diluting a suitable volume of stock solution using water. 50 μM of PFOA solution was used as a test sample to optimize the DPS-MS conditions. Great Value microwave popcorn paper and Beef Pho Instant Noodles were purchased from a local Walmart store. McDonalds' burger wrapper, Burger King wrapper, and Burger King French fry box were collected from corresponding local restaurants.

For the soil analysis, the soil sample was collected in the Keegan landfill area of New Jersey on Apr. 13, 2022. The soil sample was collected around 0.05 m deep below the ground using a steel shovel. 5 g of the soil sample was transported from the sampling location to the laboratory by depositing it into a Ziploc bag. In the laboratory, 3 g of the soil sample was measured, finely grinded, and transferred to a 20 mL glass bottle and 2 mL water was added to extract possible PFAS contaminants. The soil sample was then sonicated for 1 min in a water bath to extract PFAS.

The wastewater sample was stored in high density polyethylene (HDPE) to prevent the loss of PFAS to the container or contamination of the sample with PFAS. The wastewater sample was placed in a cooler on ice at 4° C. during transportation and was maintained at 4° C. until analysis.

A high-resolution Q Exactive Orbitrap mass spectrometer (Thermo Scientific, San Jose, CA, USA) was used to conduct MS and MS/MS experiments. The instrument was calibrated on a weekly schedule. Calibration was performed using a commercially accessible solution, Pierce™ ESI Negative Ion Calibration Solution, obtained from Thermo Scientific. The commercial ESI ion source was removed to accommodate paper spray (PS), desalting paper spray (DPS), and nano electrospray ionization (nanoESI) ion sources. The capillary inlet temperature was set to 250° C. Data analysis was acquired by Thermo Xcalibur (3.0.63). To conduct the collision-induced dissociation (CID) MS/MS experiment, 10-45 normalized collision energy (NCE) was used. Instrument parameters, such as AGC target, microscan, and maximum injection time were adjusted based on relative abundance of detected PFAS ions. In general, the AGC target was selected from 2E4˜1E6, microscans 3˜8 and maximum injection time was set from 30˜1000 ms. For the LC/MS experiment as a comparison, a Waters ultra-performance liquid chromatography (UPLC, Milford, MA, USA) system equipped with a C18 column was used for the LC/MS separation and detection of PFAS. 10 mM ammonium acetate and methanol were used as mobile phase A and B, respectively. The elution gradient was run for 18 min to separate PFAS species at a flow rate of 0.3 mL/min.

Scanning electron microscopy (SEM) analysis was performed on EM JSM-7900 F, JEOL operated at 15 kV to characterize the surface morphology. SEM micrograms of target samples were taken at ×200 magnification. Energy-dispersive X-ray spectroscopy (EDX) was used to analyze the elemental composition of the paper filter before and after loading PFAS.

Referring to FIG. 1A, to conduct PS-MS analysis in the Examples, Fisher brand qualitative p8 filter paper was cut into triangular shapes (10 mm×5 mm, height×width), and cleaned by sonicating sequentially by acetone, methanol, and methanol/water (50:50 v/v, 15 min each). A sample solution was applied to the cleaned filter paper. In some Examples, 10 μL of sample was added 4 times onto the cleaned filter paper to enhance the detection sensitivity. After drying, the filter paper was held in front of the MS inlet (8 mm away) using a high-voltage cable with an alligator clip, and 30 μL of MeOH (100%) was added directly onto the filter paper to elute target compounds for ionization upon application of a high voltage (−3.5 kV) to the wetted filter paper.

Referring to FIG. 1B, to conduct DPS-MS analysis in the Examples, Fisher brand qualitative p8 filter paper (Fisher Scientific) was cut and cleaned using the same process as PS-MS analysis described above for FIG. 1A. A sample solution was applied to the cleaned filter paper, which was placed on top of a Kimwipe to facilitate the absorption by capillarity. In some Examples, 10 μL of sample was added 4 times onto the filter paper to enhance the detection sensitivity. Desalting was achieved by loading 30 μL of ultrapure H₂O (10 μL of H₂O each time, three loadings) onto the filter paper placed on top of another Kimwipe (to facilitate absorption of the water by capillarity) to wick the sample salts and other matrix chemicals. The filter paper was then held in front of the MS inlet (8 mm away) using a high-voltage cable with an alligator clip, and 30 μL of MeOH (100%) solution was applied to the filter paper to elute the target compounds for spray ionization upon application of a high voltage (−3.5 kV) to the wetted filter paper.

To conduct nanoESI-MS analysis in the Examples, a pulled fused-silica capillary with a conical tip (i.d., ˜1 μm) was produced using a laser puller (model P 1000, Sutter Instrument Inc., USA). The sample solution was directly infused into the pulled fused-silica capillary at a flow rate of 2 μL/min using a syringe driven by a syringe pump. The capillary was placed 8 mm distance from the mass spectrometer inlet, and high voltage (−3.5 kV) was applied to the syringe needle to trigger ionization.

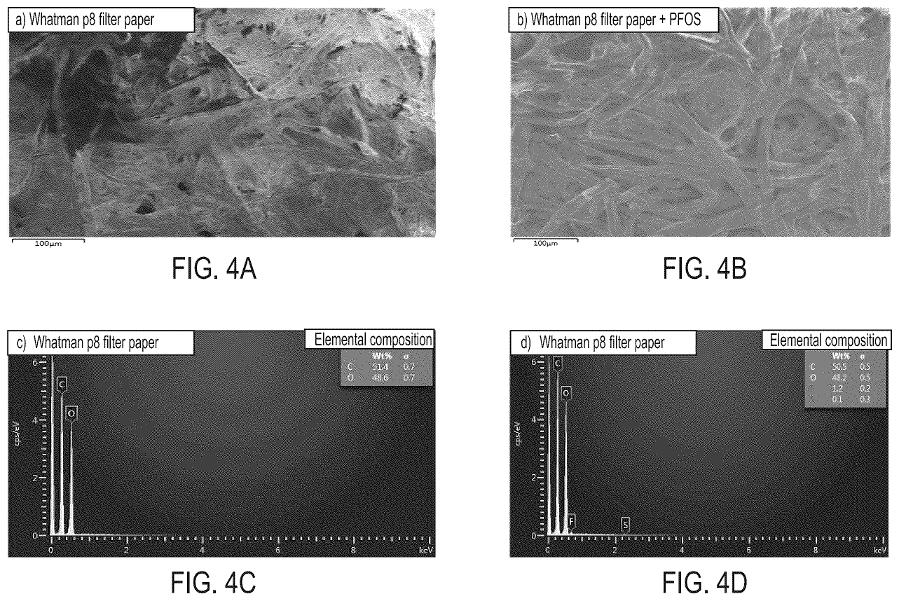

Because PFAS is ubiquitous, care was first taken to ensure no PFAS signal was seen from a blank control experiment. In the blank control experiment, a triangular-shaped blank filter paper was examined for cross-contamination of the most pervasive PFOA, PFOS, PFHxA and PFBS. For this experiment, a blank paper was attached to a high voltage alligator clip, which was aligned and held in front of the mass spectrometry inlet, and 30 μL of MeOH was added directly to the blank filter paper to extract PFAS for ionization and detection. No PFOA, PFOS, PFHXA and PFBS were detected in this control experiment (MS spectrum shown in FIG. 5).

### Example 1—DPS-MS Signal Optimization

In some preliminary tests using the nanoESI-MS methods described herein, a signal suppression effect was found for samples containing high levels of non-volatile salt matrices. To solve this problem, an “on-paper” desalting step (i.e., a desalting solvent was applied directly to the filter paper having the sample loaded thereon to remove salts) was added to the PS-MS methods, namely a DPS-MS method was carried out for PFAS analysis.

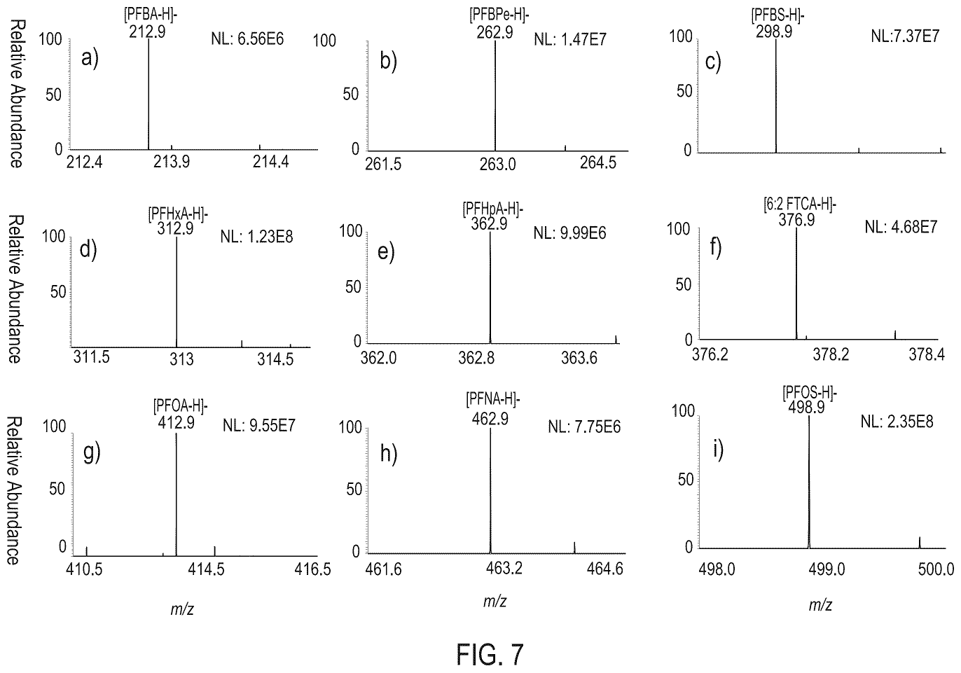

Tests were carried out to optimize the parameters of a DPS-MS signal, using 50 μM PFOA solution prepared in 50 mM KCl as the test sample. The ion signal from DPS ionization could be influenced by various factors, including the distance between the filter paper and the mass spectrometry inlet, the volume of the loaded sample, the amount of spray solvent (elution solvent) used, the applied spray voltage, the elution solvent, and the number of desalting cycles. To optimize the distance between the filter paper and MS inlet, three different distances, 4, 8, and 12 mm were tested. It was found that the highest intensity of PFOA at m/z 412.9 was 3.77E8 at 8 mm distance and ion signal decreased with increasing distance (FIG. 8A).

Sample loading volume onto the filter paper was analyzed by loading various sample volumes from 4 to 10 μL. It was demonstrated that the highest signal intensity was at 10 μL sample loading (FIG. 8B). While a single application of a 10 μL sample was determined to provide the highest signal intensity for a single application, multiple applications of 10 μL sample (e.g., application of 10 μL sample four times) can further enhance the ion signal as further described herein.

The spray solvent (elution solvent) volume effect was also examined for extraction and ionization of PFOA, by increasing the volume applied beginning at 10 μL. Signal intensity increased slightly from 10 μL to 20 μL, with a significant increase in signal intensity with application of 30 μL elution solvent (FIG. 8C).

Analysis of spray voltage was carried out starting with a negative voltage of 2.5 kV, which showed minimal signal intensity. An increase of negative voltage to 3 kV increased the signal intensity, as well as a further increase in intensity when the negative voltage was raised to 3.5 kV. Increasing the negative voltage beyond 3.5 kV to 4 kV resulted in a decline in signal intensity. Thus, the peak ion signal intensity was seen at a negative voltage of 3.5 kV, as illustrated in FIG. 8D.

To analyze the effect of the type of elution solvent used, H₂O, ACN and MeOH were examined, with MeOH demonstrating the highest intensity (FIG. 8E).

The number of desalting cycles applied (10 μL H₂O was used each time) was further analyzed to determine the optimal conditions for removal of ion suppression metrices. As illustrated, the highest signal intensity (i.e. least amount of signal suppression) was demonstrated with three cycles of 10 μL H₂O (FIG. 8F).

Based on these studies, the optimized experimental conditions were determined to include: distance between filter paper and MS inlet (8 mm); spray ionization voltage (−3.5 kV), elution solvent (MeOH), sample loading volume (10 μL); spray solvent (elution solvent) volume (30 μL MeOH), and number of desalting cycles (three times). Thus, in the Examples below, these optimized experimental conditions were utilized for DPS-MS unless otherwise noted. It is noted that these optimized experimental conditions also apply to the PS-MS methods described herein with the absence of the desalting steps. Thus, in the Examples below, these optimized experimental conditions were utilized for PS-MS unless otherwise noted.

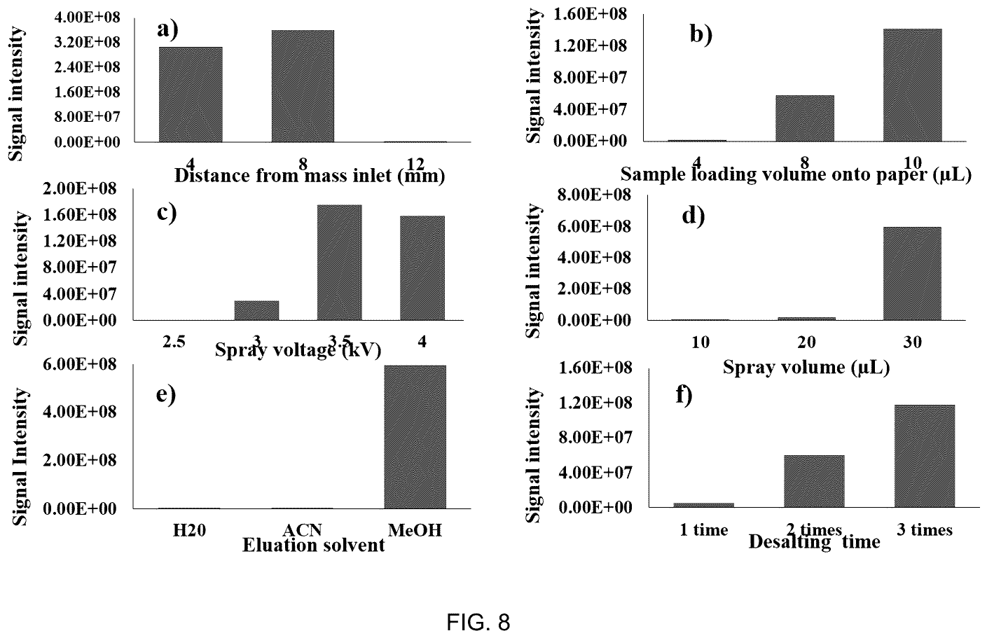

Signal stability was also checked for PFOA at m/z 412.9 in the DPS-MS experiment. After spraying the solvent of MeOH, when the potential was applied at t=0 min, there was a time delay of about 0.13 min to obtain a stable EIC current for m/z 412.9, as illustrated in FIG. 9. The delay might have been caused by the electrophoretic migration of the PFOA sample to the filter paper spray tip. The signal stabilized for about 0.42 min (0.13-0.55 min) before declining. It is noted that the EIC peak of DPS-MS was wide and not as symmetrical as a typical LC/MS EIC peak, but the signal lasted a long period of time which would favor collection of MS/MS data.

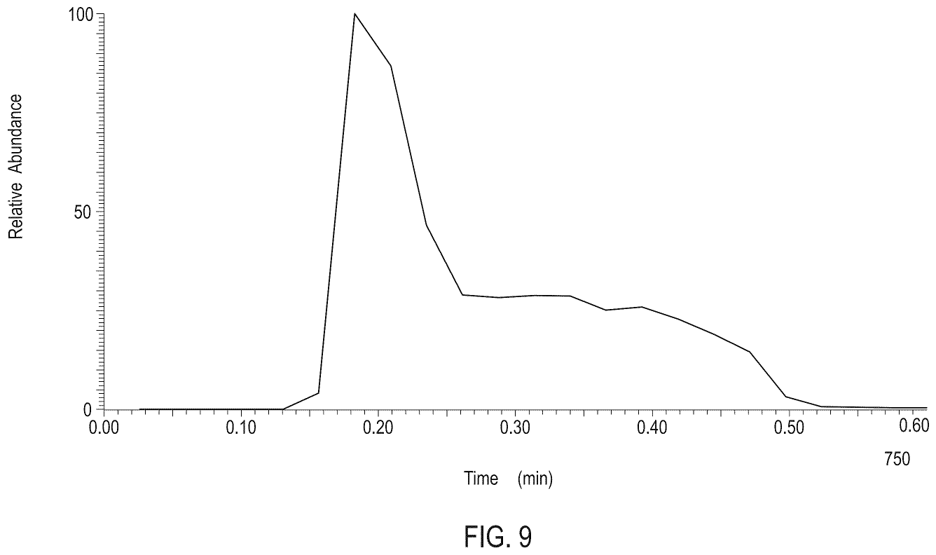

To perform a recovery test, a 50 μM PFOA sample was prepared without using KCl (50 mM). This sample was tested first, with 10 μL of the sample directly applied onto a triangular-shaped filter paper and eluted by MeOH for ionization. For comparison, a second 50 μM PFOA sample was prepared in 50 mM KCl. The KCl matrix was removed (or at least partially removed) from the PFOA sample by washing with 10 μL H₂O three times, and then was subjected to MS detection during DPS-MS analysis. Both samples were tested three times. Results were averaged and shown in FIG. 10. The intensity of PFOA without 50 mM KCl at m/z 412.9 was 1.93E8. The signal intensity for PFOA with 50 mM KCl (washed with H₂O) was 1.75E8. The signal ratio of two signal intensities was 91%, indicating the PFAS recovery of 91% after water washing for desalting. This result confirmed that PFOA has a strong interaction with the filter paper and allows the present desalting techniques to remove ion suppression matrices while maintaining PFOA on the filter paper.

### Example 2—DPS-MS Analysis Using PFAS Stock Solutions

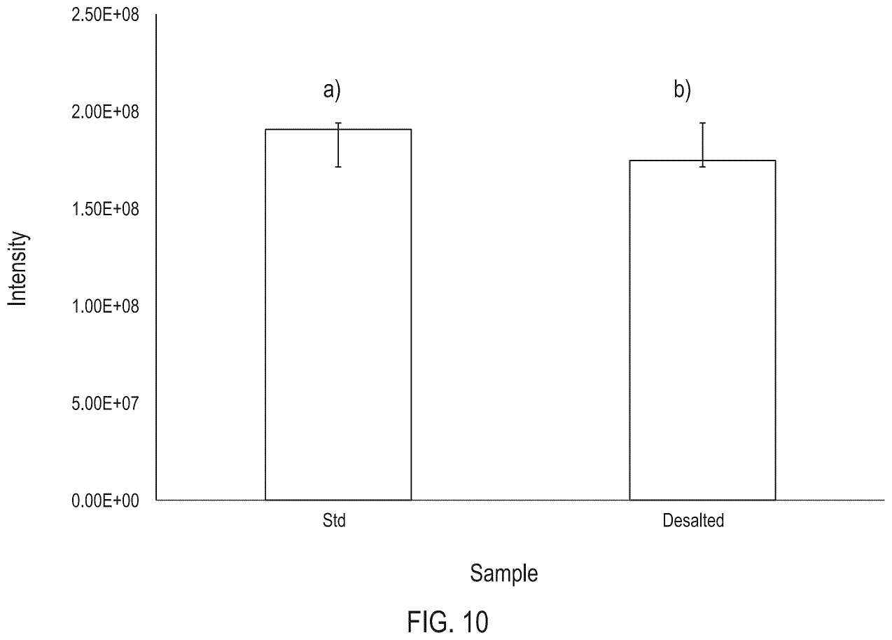

After signal optimization, an analysis of PFOA was conducted by the present DPS-MS method and was compared to traditional nanoESI-MS. First, 50 μM PFOA in 50 mM KCl in H₂O was directly ionized by nanoESI, as shown in the MS spectrum (FIG. 11A). As illustrated, an ion C₇F₁₅COO— at m/z 412.9 (measured m/z 412.96482, theoretical m/z 412.96643 and mass error: −3.9 ppm) corresponding to deprotonated PFOA was detected but the intensity was low (7.88E4). The low intensity of the PFOA ion may be due to ion suppression by the KCl salt matrix. By adding MeOH to the sample (final volume ratio of MeOH to H₂O:1:1), the ion signal was improved to 2.04E6 (FIG. 11B).

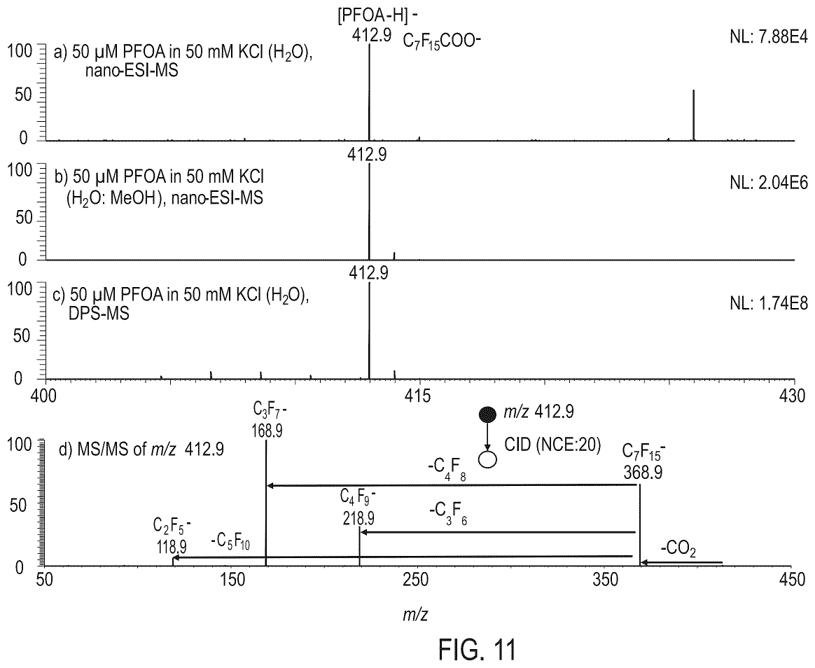

In contrast, when 50 μM PFOA aqueous solution containing 50 mM KCl was ionized by DPS-MS (via desalting on the filter paper using water, followed by MeOH elution for ionization spray under high voltage), the PFOA signal was 1.74E8, which was more than 80-2000 times higher than that from the nanoESI-MS technique. FIG. 12 illustrates the corresponding EIC spectra of the PFOA ion [PFOA-H]-(m/z 412.9) detected by nanoESI and by DPS-MS according to the present methods. As demonstrated, the nanoESI ion signal was not very stable, probably due to the salt present in the samples. DPS-MS was demonstrated to be clearly superior to nanoESI for PFOA analysis in salt-containing samples, due to the capability of removing the salt matrix. In addition, DPS-MS is fast (about 3 min per sample), as desalting and spray ionization are performed using the same filter paper. The assignment of the PFOA ion from DPS-MS was further confirmed through CID analysis. Upon CID, the ion at m/z 412.9 gave rise to fragment ions of m/z 368.9, 218.9, 168.9, and 118.9, by consecutive losses of CO₂ and C₃F₆, C₄F₈ or C₅F₁₀ (FIG. 11D) consistent with its structure.

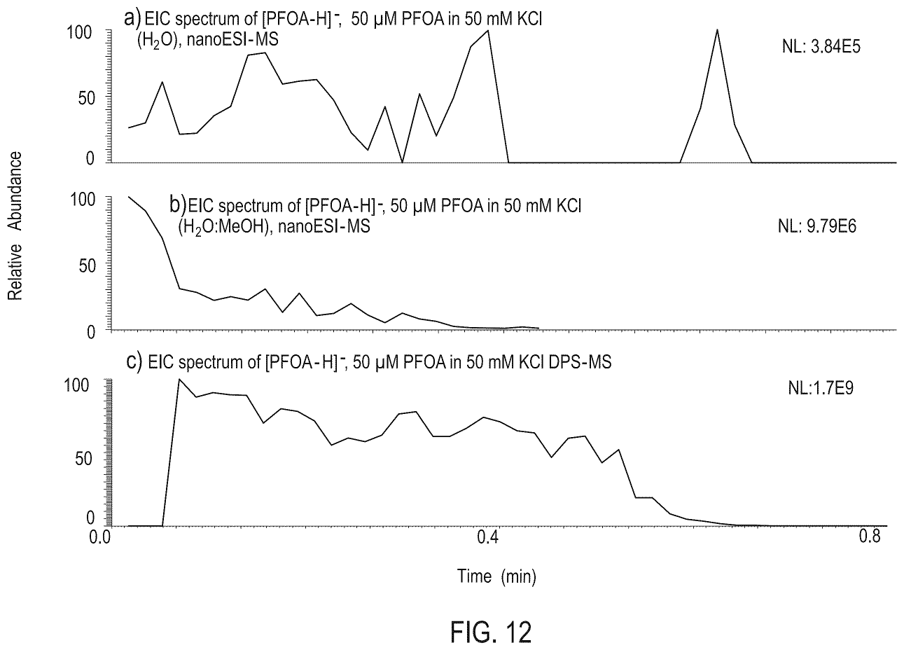

Another set of experiments was conducted to determine if the DPS-MS method could tolerate other salt matrices to detect PFAS in a low concentration. PFOA (1 nM) was dissolved in 50 mM NaCl and 50 mM Na₂SO₄ and analyzed by DPS-MS according to the described methods. The signal of PFOA at m/z 412.9 was clearly detected in the presence of both salts within mass error<4 ppm (FIGS. 13B and 13D). In contrast, nanoESI-MS failed to detect PFOA in the presence of salt NaCl or Na₂SO₄ (FIGS. 13A and 13C).

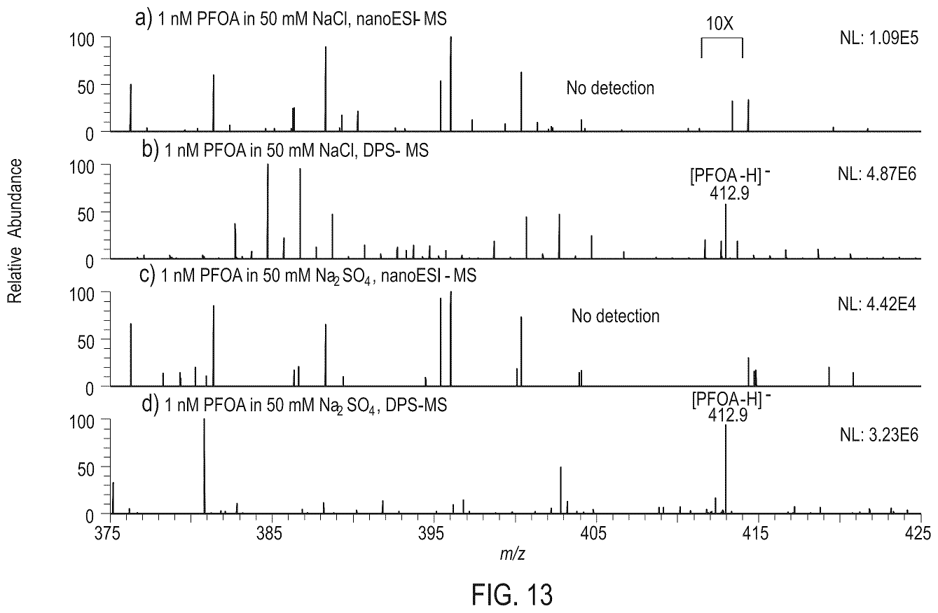

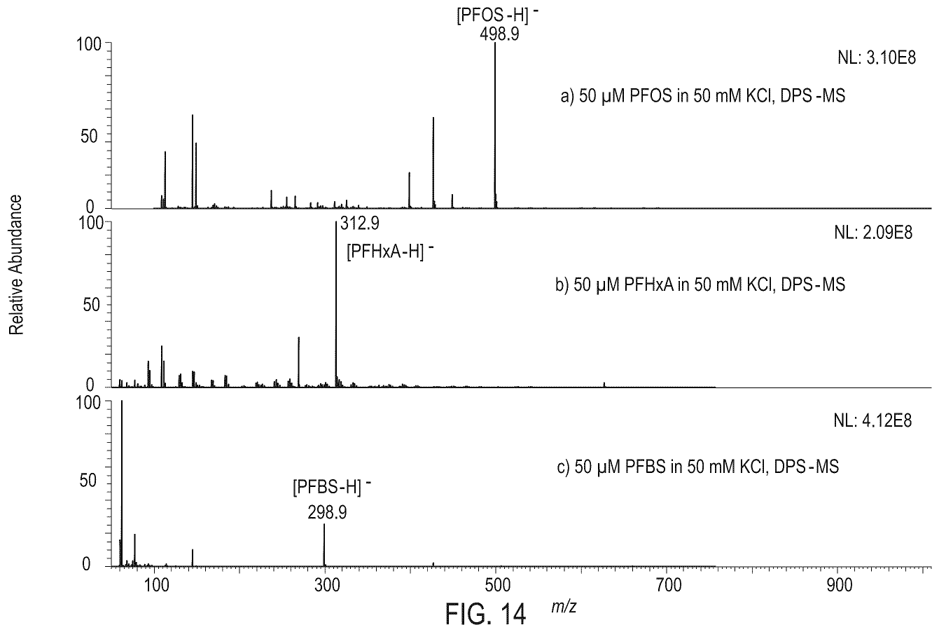

The application of DPS-MS was further examined to analyze other PFAS, including PFOS, PFHxA and PFBS (structures shown in Table 1, FIG. 39). As shown in FIG. 14, 50 μM PFOS, PFHxA and PFBS in water containing 50 mM KCl were analyzed by DPS-MS and all these PFAS were clearly detected with high ion intensity (>10E8). Similar to the deprotonated PFOA (m/z 412.9), upon CID, these PFAS ions lose either CO2 or SO3 and undergo C—C bond cleavages by losses of C2F4, C3F6, etc. (FIG. 15, and Table 1), consistent with their ion assignments. These results demonstrate that DPS-MS is capable of detecting PFAS in the presence of salt matrices, in general.

### Example 3—Interaction of PFAS With Filter Paper for DPS-MS

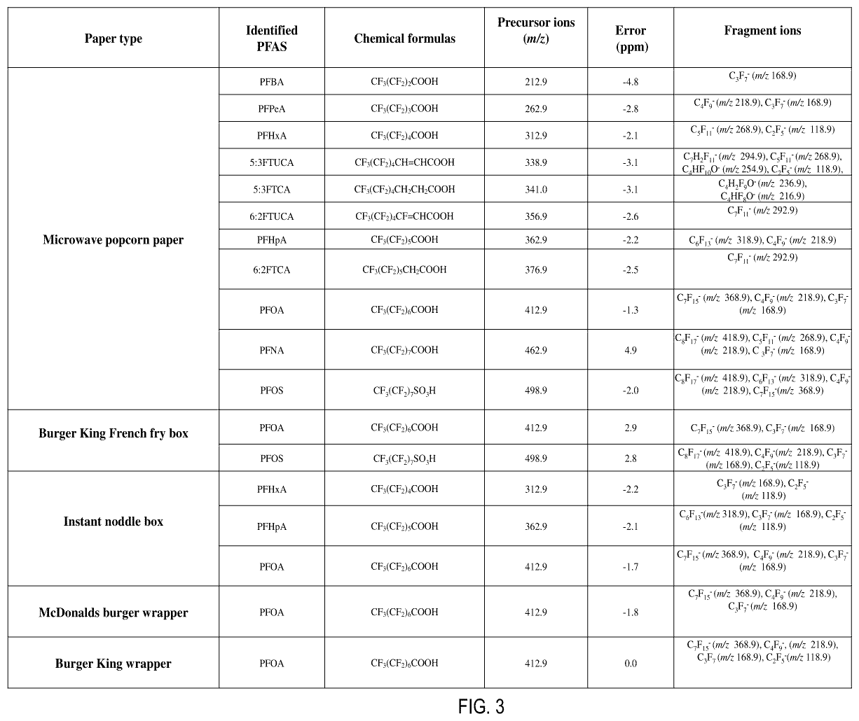

The interaction of PFAS with filter paper used for DPS-MS methods was analyzed. As previously noted, PFAS molecules often contain a hydrophobic tail, such as perfluoroalkyl groups CnF2n+1 and a hydrophilic polar head such as CO₂_{^{−}} or SO₃_{^{−}}. Due to the possible polar-polar interactions between the polar head of targeted PFAS and cellulose filter paper, it was believed that PFAS would be retained on the filter paper upon addition of water to wash away the salt matrix. To confirm the retention of PFAS on the surface of desalted filter paper, SEM and EDX investigations were conducted. The SEM micrograph evidenced the fibrous nature of the filter paper surface with the presence of pores (FIG. 4A). The porous nature of the surface allowed the removal of salt matrices by water rinsing. Upon applying the PFOS to the filter paper surface, the pores became smeared (FIG. 4B), suggesting the adsorption behavior of PFOS, or binding of PFOS with the filter paper surface. The EDX spectrum of the filter paper showed the existence of fluorine (F) and sulfur (S) peaks (FIG. 4D). Such EDX data confirmed the adsorption of PFOS on the filter paper, as PFOS is the source for fluorine and sulfur.

In contrast, the blank filter paper showed only the elemental composition of paper, consisting of carbon (C) and oxygen (O) (FIG. 4C). Also, the relative elemental compositions of carbon and oxygen decreased following the adsorption of PFOS as shown in FIGS. 4C and 4D. It has been established that the sulfuric group of PFOS is negatively charged and hydrophilic. On the other hand, the C−F chain of PFOS is more hydrophobic. Besides these polar-polar group interactions, the hydrophobicity of C−F enhanced intermolecular interaction of PFOS may also contribute to stabilizing PFOS adsorption onto the filter paper (e.g., see Yeung L W Y, De Silva A O, Loi E I H, Marvin C H, Taniyasu S, Yamashita N, Mabury S A, Muir D C G, Lam P K S, Perfluoroalkyl substances and extractable organic fluorine in surface sediments and cores from Lake Ontario. Environment International. 2013; 59:389-397).

### Example 4—PFAS Detection Sensitivity of DPS-MS

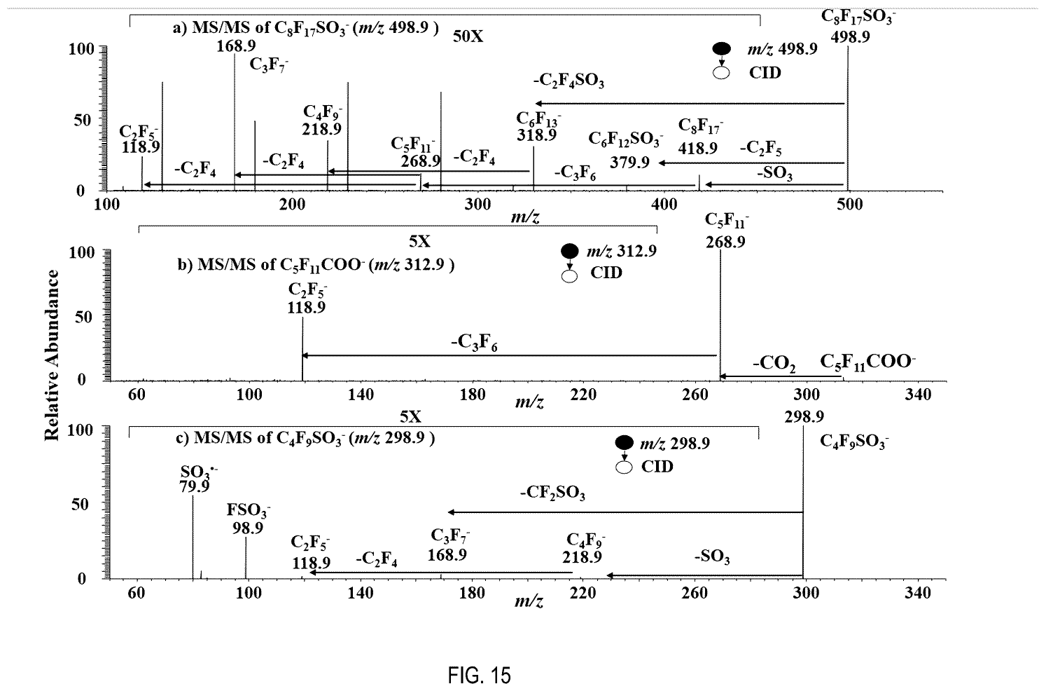

The PFAS detection sensitivity of the present DPS-MS methods was evaluated. For this experiment, samples containing a constant concentration of KCl (50 mM) were used, while the concentration of PFOA, PFOS, PFHxA, and PFBS were reduced from 50 μM to 100 pM so that the KCl concentration was 5×10⁸ times higher than the PFAS. As illustrated in FIG. 16, all four PFAS ions were successfully detected (PFOA: measured m/z 412.96780, theoretical m/z 412.96643, mass error: 3.3 ppm; PFOS: measured m/z 498.93094, theoretical m/z 498.93022, mass error: 1.4 ppm; PFHxA: measured m/z 312.97278, theoretical m/z 312.97281, mass error: −0.1 ppm; PFBS: measured m/z 298.94292, theoretical m/z 298.94299, mass error: 0.2 ppm), despite the presence of a high concentration KCl matrix. In contrast, the nanoESI-MS failed to detect these PFAS ions.

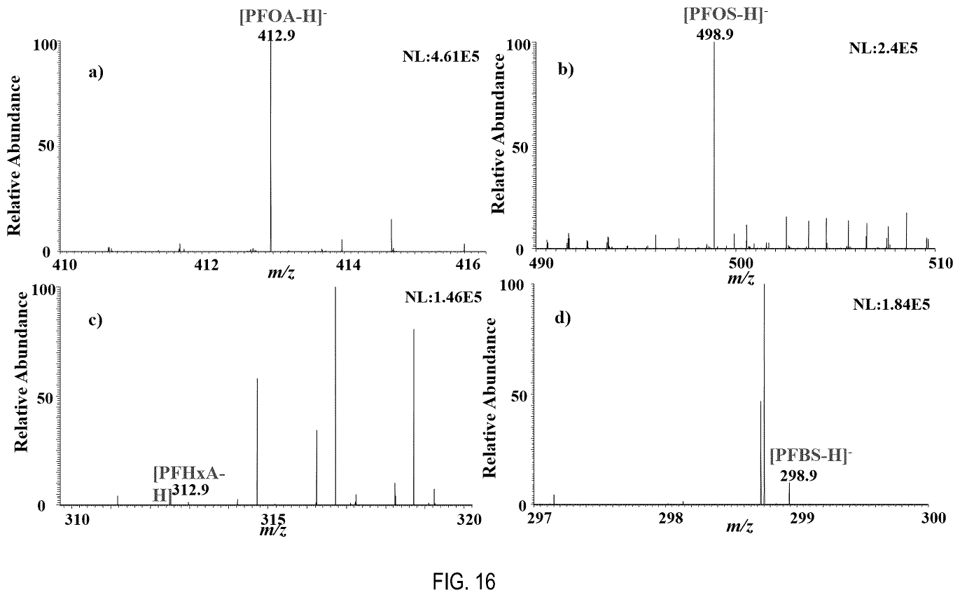

In the case of PFOA, the PFOA concentration was lowered 10 times more to evaluate the DPS-MS sensitivity. A 10 PM (4.1 ppt) PFOA sample in 50 mM KCl (KCl concentration was 5×109 times higher than PFOA) was prepared. In addition, a control experiment was conducted in parallel by adding 50 mM KCl onto filter paper followed by filter paper washing, in which no PFOA was detected (FIG. 17A). As shown in FIG. 17B, the PFOA signal was clearly observed with sufficient mass error (measured m/z 412.96770, theoretical m/z 412.96643, mass error: 2.4 ppm) using the DPS-MS technique. Therefore, the present DPS-MS was demonstrated to provide a desalting capability and, at the same time, high sensitivity for PFAS analysis.

### Example 5—DPS-MS Soil Sample Analysis vs. PS-MS Soil Sample Analysis

The DPS-MS method of the present disclosure was applied for PFAS analysis of a soil sample extract. To conduct the experiment, a 40 μL soil sample extract (aqueous) was directly added onto filter paper and washed with 30 μL water (desalting) to remove the background matrices. A parallel experiment was conducted by adding the same soil sample extract and sample amount for PS-MS analysis without the desalting step.

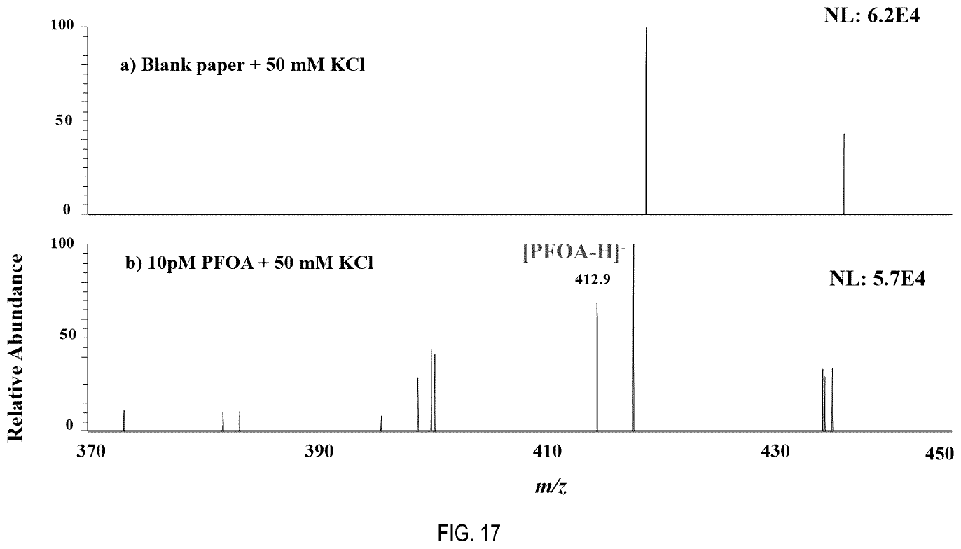

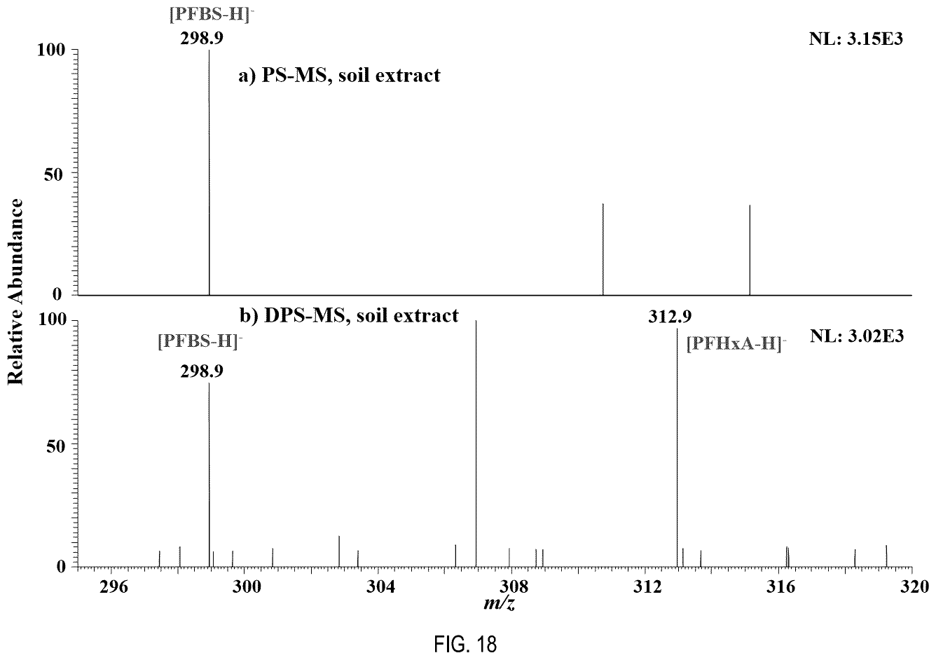

As shown in the corresponding spectra (FIG. 18), while PFBS at m/z 298.9 was detected in both the DPS-MS and PS-MS (without desalting) experiments with similar intensity, PFHxA m/z 312.9 was only detected by DPS-MS with good mass accuracy (measured m/z 312.97170, theoretical m/z 312.97281, mass error: −3.5 ppm). The results suggest that the background matrices of the soil extract may have caused signal suppression in the PS-MS experiment, while the washing step during the DPS-MS analysis removed the background matrices. Initially detected PFAS was confirmed by a collision-induced dissociation MS/MS study (illustrated in FIG. 19). It is noted that due to low intensity of PFHxA and PFBS, only one fragment ion was observed during the CID measurement.

### Example 6—Quantitative DPS-MS Soil Sample Analysis

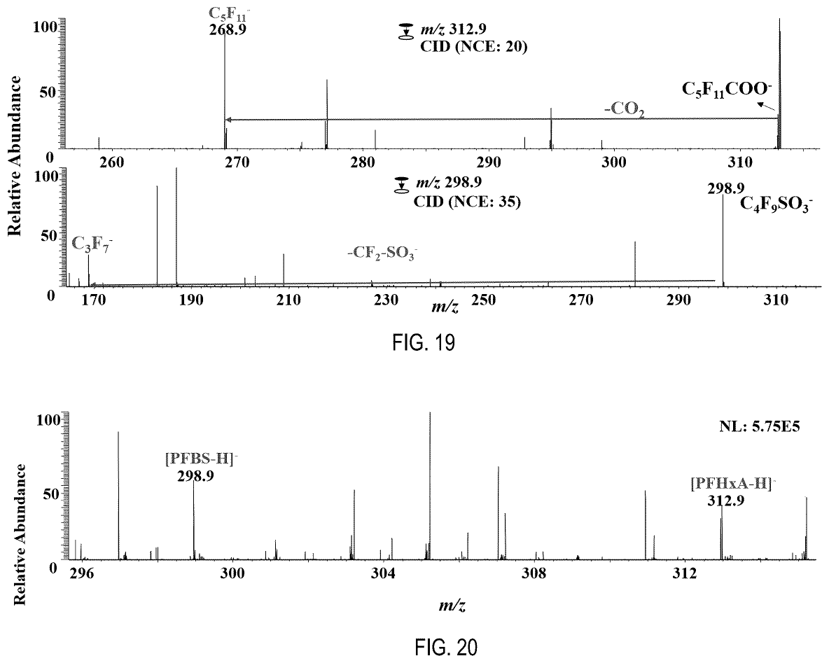

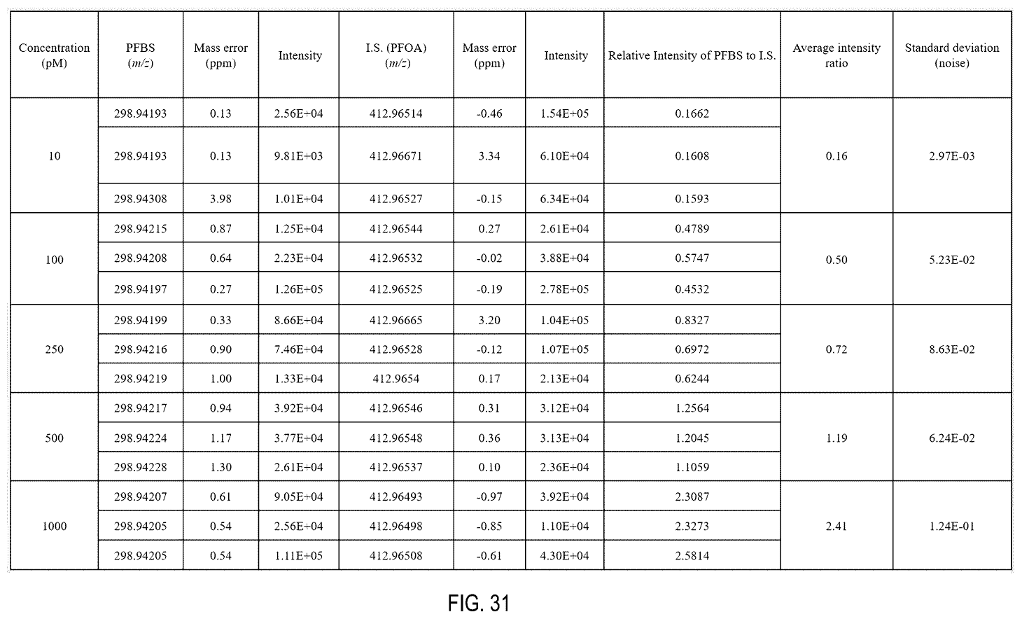

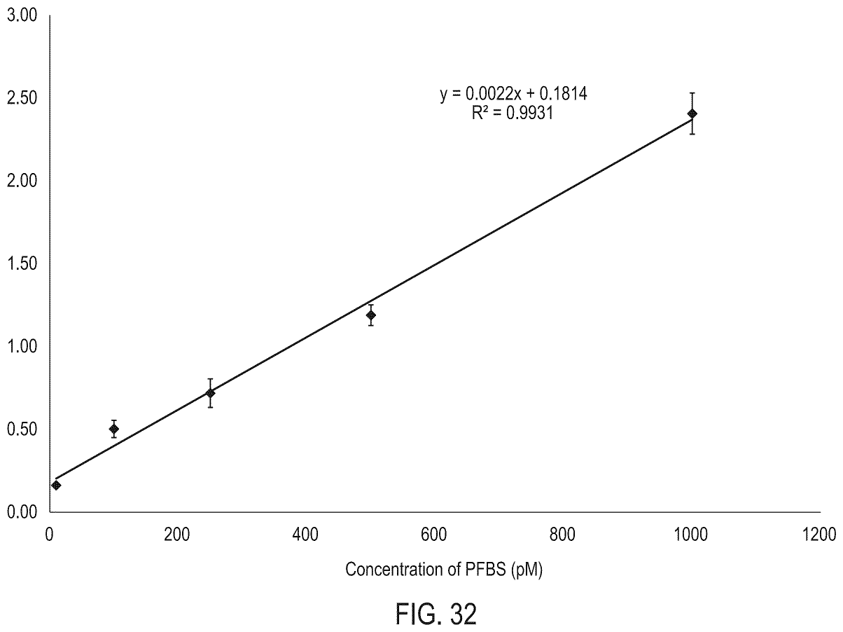

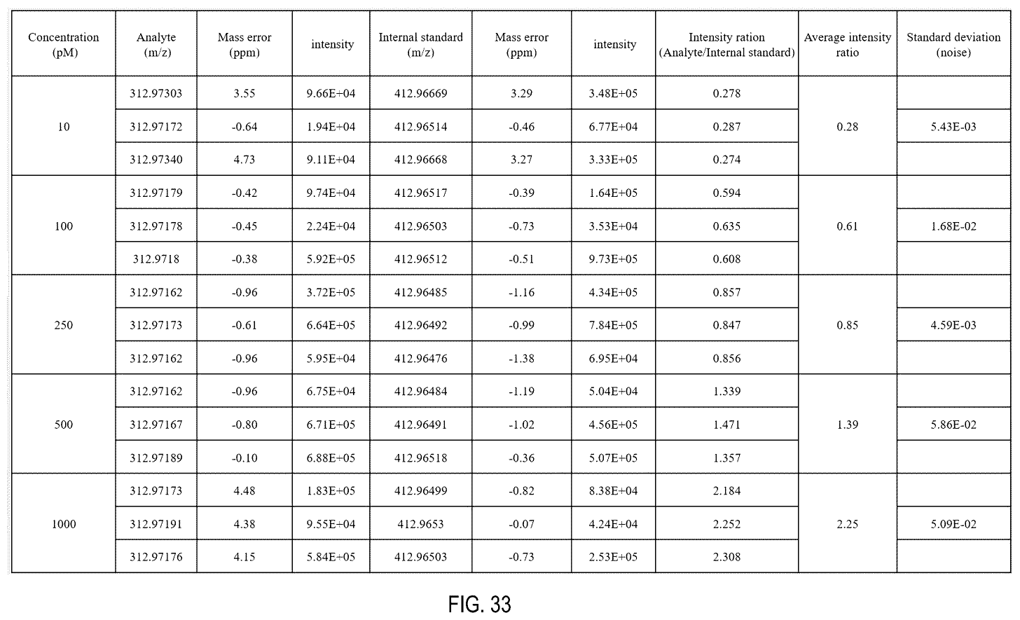

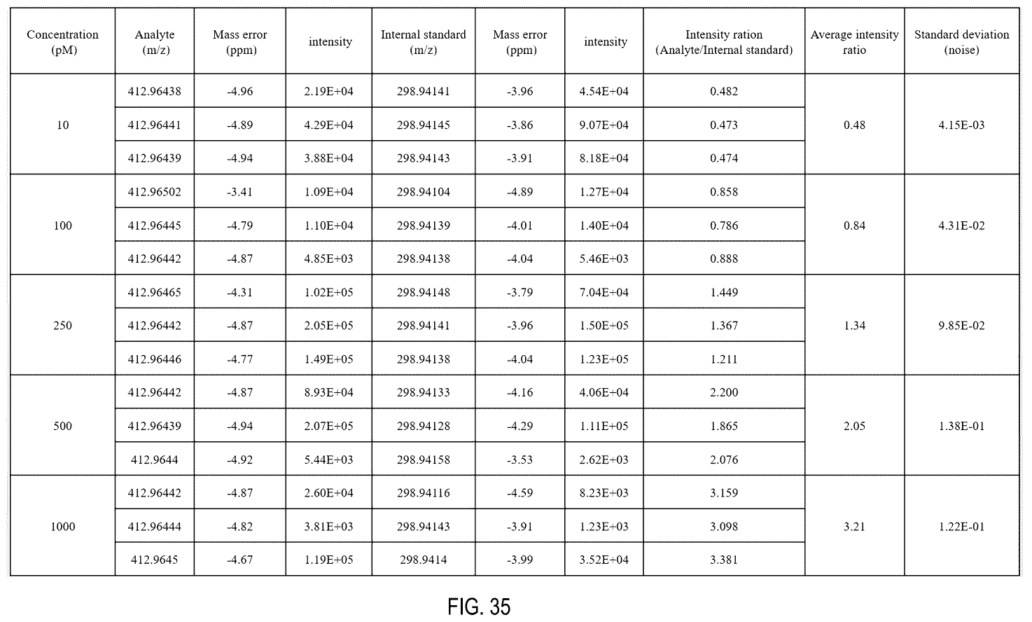

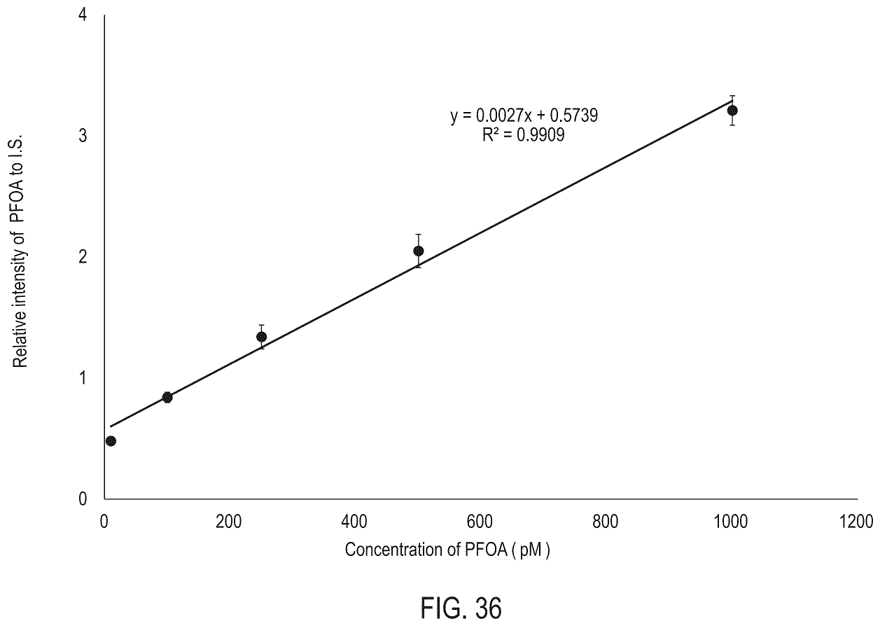

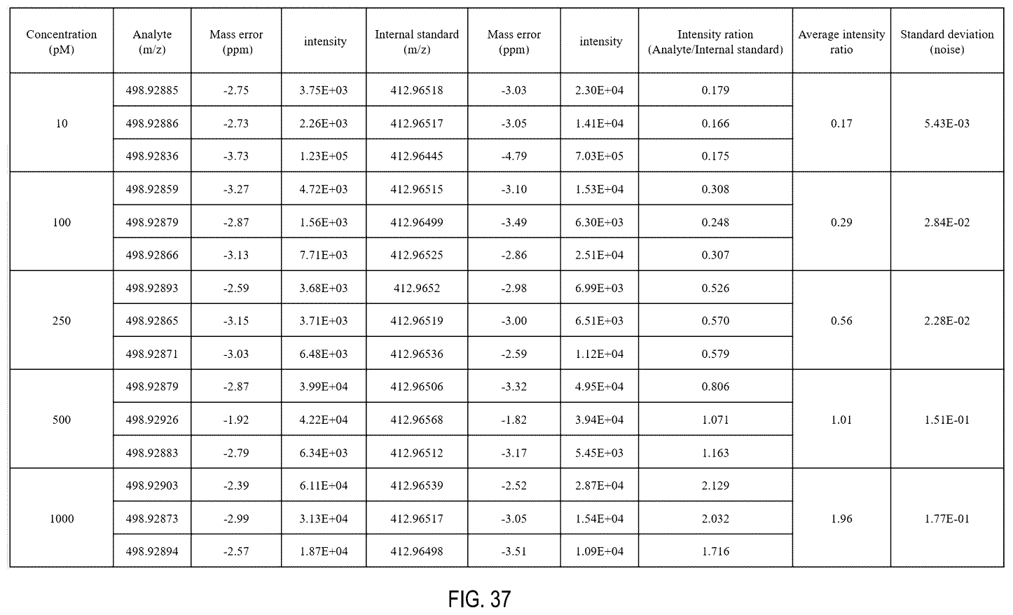

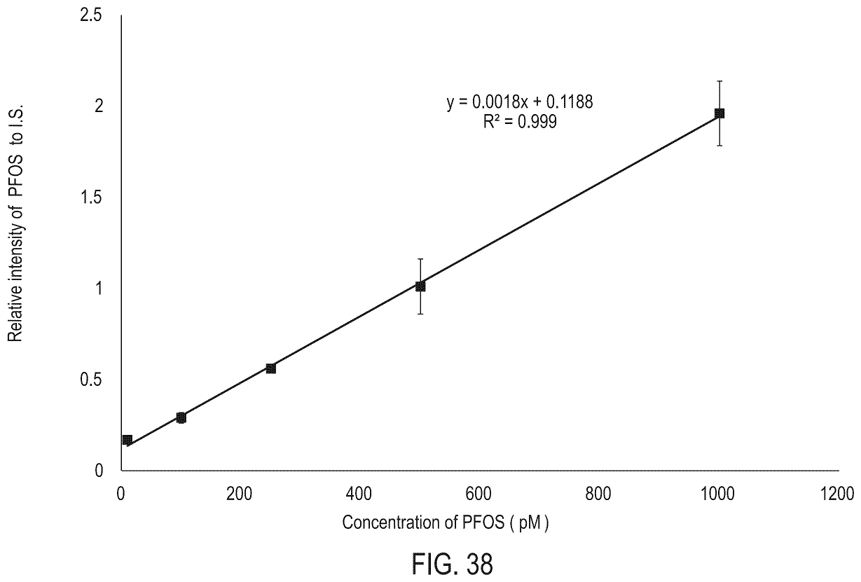

To conduct quantitative experiments, PFOA was doped as an internal standard (IS). Initially, a 40 μL soil sample extract (doped with 400 pM PFOA) was applied to the filter paper, and the mass spectrum was acquired by the present DPS-MS method, which showed the detection of PFBS (m/z 298.9) and PFHxA (m/z 312.9, FIG. 20). To measure the amounts of PFBS and PFHxA in the soil extract sample, calibration curves of PFBS (FIG. 32) and PFHxA (FIG. 33) were made (Raw data are shown in Table 3, FIG. 31 and Table 4, FIG. 33, respectively). The limit of detection (LOD) for PFBS and PFHxA was calculated as 1.2 and 2.7 ppt, respectively. The amounts of PFBS and PFHxA in the soil extract were quantified and found to be 145.2 and 79.3 ppt, respectively. To achieve reproducible quantitative analysis results, filter paper substrates of the same size were adopted. The same volume (10 μL) of sample was added onto the same location of each filter paper. In addition, the filter paper position was fixed relative to the MS inlet using a xyz stage platform. A calibration curve of PFOA (FIG. 36, based on the raw data provided in FIG. 35) and PFOS (FIG. 38, based on the raw data provided in FIG. 37) was also made at various concentrations and the LODs of PFOA and PFOS were found to be 1.9 and 4.5 ppt.

To address any potential issue of PFAS dilution during the extraction process, an alternative approach was pursued by conducting direct DPS-MS analysis of soil samples (without prior solvent extraction). This method offers advantages such as additional time-saving and the ability to use a smaller amount of sample (e.g., 40 mg in this analysis). Typically, the detection of PFAS in soil samples involves labor-intensive sample preparation techniques that include solvent extraction followed by solid-phase extraction to concentrate PFAS.

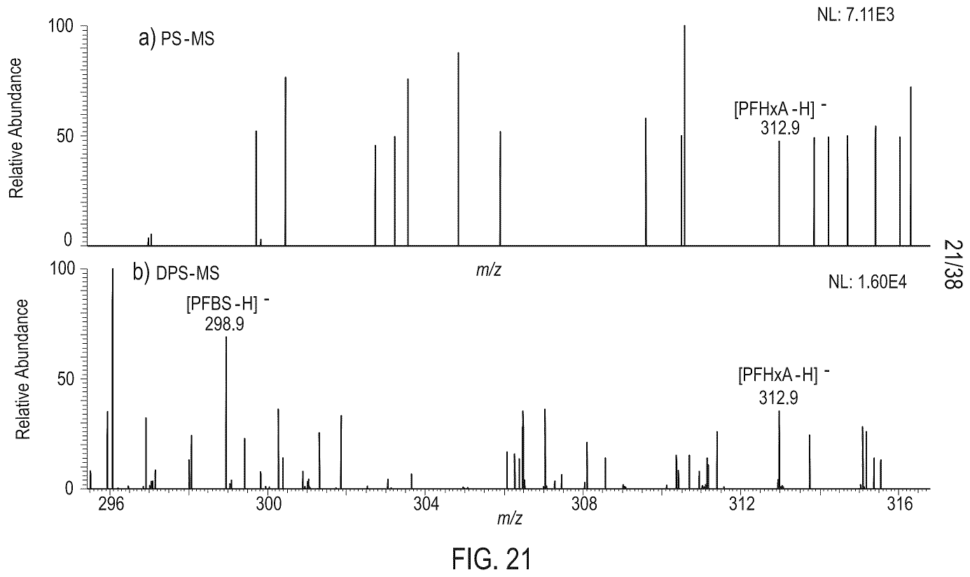

First, PS-MS was applied, and the deposited soil sample was directly ionized with MeOH (100%) applied to the soil sample for paper spray ionization using an applied high voltage of −3.5 kV. FIG. 21A shows detection of PFHxA with low signal intensity (3.03E3), which might be due to interference of background matrices.

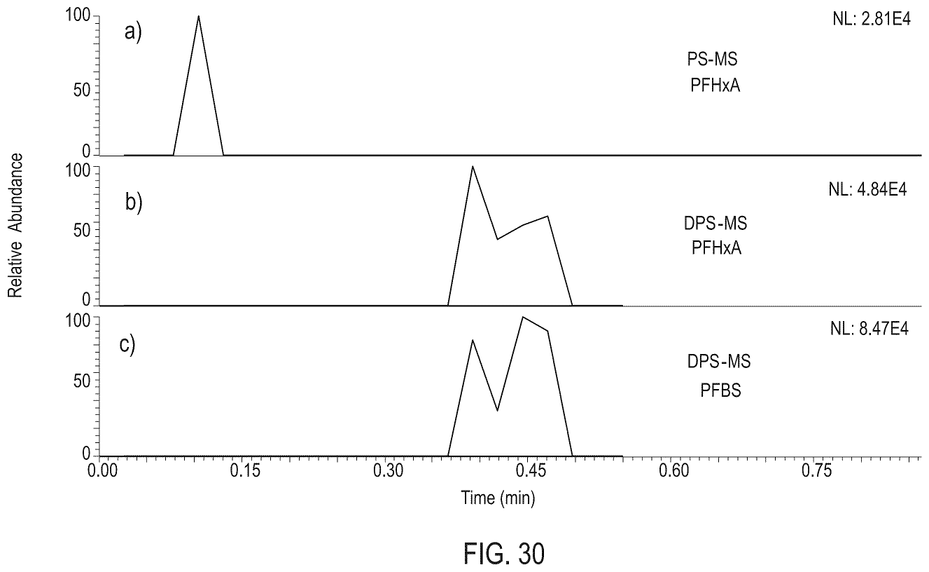

DPS-MS was then applied by adding water (as a desalting material) to the soil sample on the filter paper, followed with spray ionization using MeOH (100%) and a voltage of −3.5 kV. As illustrated, both PFBS and PFHxA were detected with elevated intensity (1.14E4 and 5.72E3) as compared to the PS-MS method without desalting (FIG. 21B). EIC spectra of the detected ions by PS-MS and DPS-MS are shown in FIG. 30.

It was demonstrated that the DPS-MS methods effectively streamline the analysis process and mitigate challenges associated with matrix interference in soil samples when detecting PFAS. It was further demonstrated that the desalting step in DPS-MS played an important role during the soil analysis as it cleaned up background interference and boosted the PFAS signal intensity.

### Example 7—Identification and Measurement of PFAS in Various Water Samples

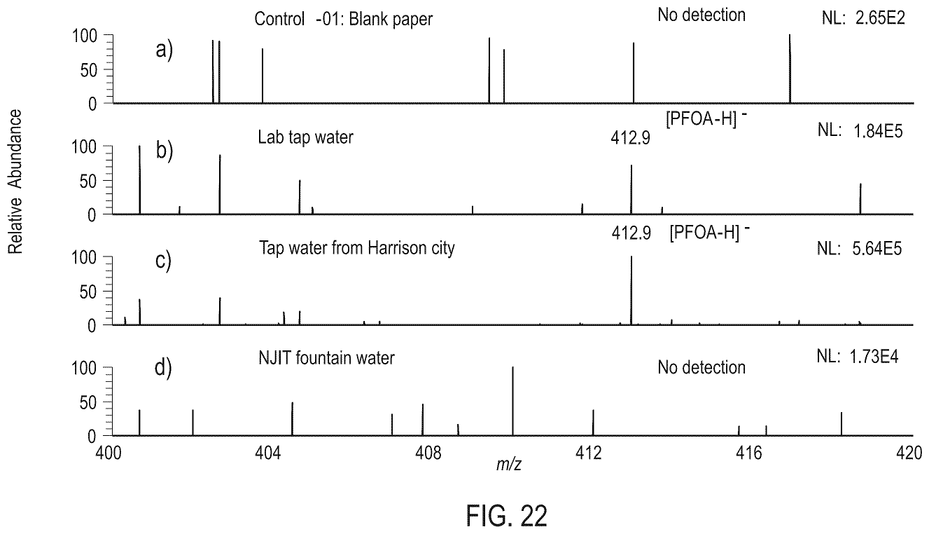

To conduct water analysis, water samples were collected from three different locations: Harrison City (Harrison, NJ), New Jersey Institute of Technology (Newark, NJ), and an anonymous location. After collecting water, a 10 μL sample was added to the filter paper four times, and 30 sec was allowed to pass for drying. As shown in the acquired MS spectra (FIGS. 22B and 22C), PFOA with low intensity was detected in both the Harrison and NJIT tap water samples (mass error <2.6 ppm), while no PFOA was observed in a blank filter paper sample (FIG. 22A). Interestingly, no PFOA was found in NJIT fountain water (FIG. 22D), possibly due the filtration system at NJIT.

To test the accuracy of the present PS-MS method for PFAS detection, the NJIT lab tap water sample was spiked with low concentrations of PFBS, PFHxA, and PFOS (100 ppt each). This NJIT lab tap water sample only contained PFOA before spiking (FIG. 22B). After spiking, all three spiked PFAS were detected along with the pre-existing PFOA by PS-MS, which demonstrates the reliability and accuracy of the present PS-MS detection of PFAS in real samples.

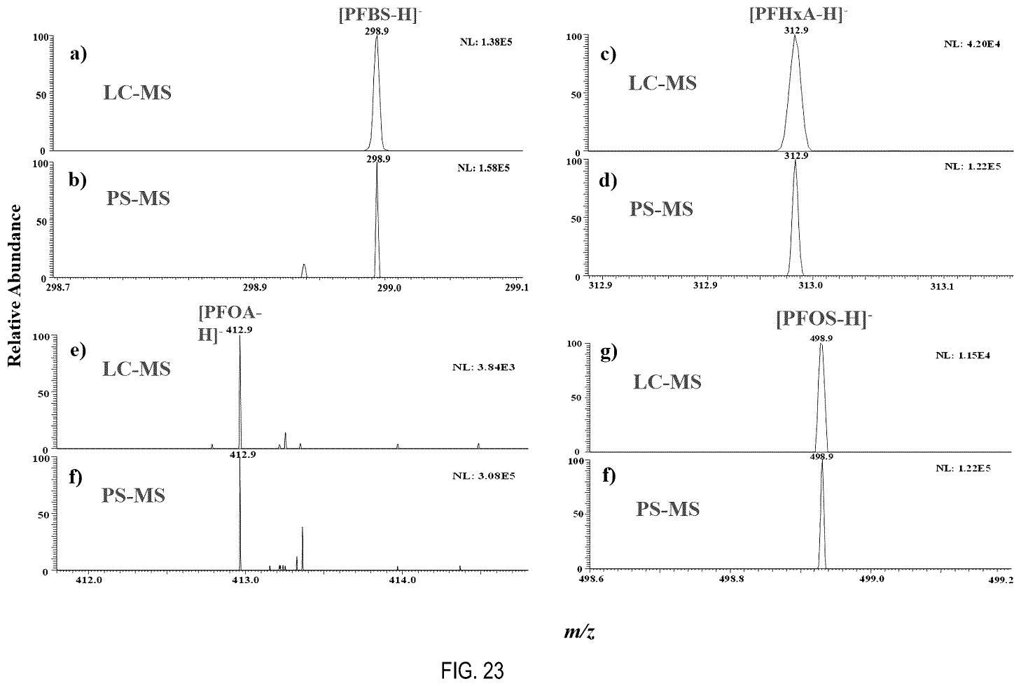

In comparison, LC/MS was conducted for the spiked NJIT tap water sample. Although all three spiked PFAS along with PFOA were detected by LC/MS, their ion intensities from LC/MS were lower than those detected by the present PS-MS methods (FIG. 23). In particular, the ion intensities for PFOS and PFOA from LC/MS was lower than those of PS-MS by 10 and 88 folds, respectively. This result demonstrates the strength of the present PS-MS methods to detect trace amount of PFAS in real samples.

### Example 8—Identification and Measurement of PFAS in Wastewater

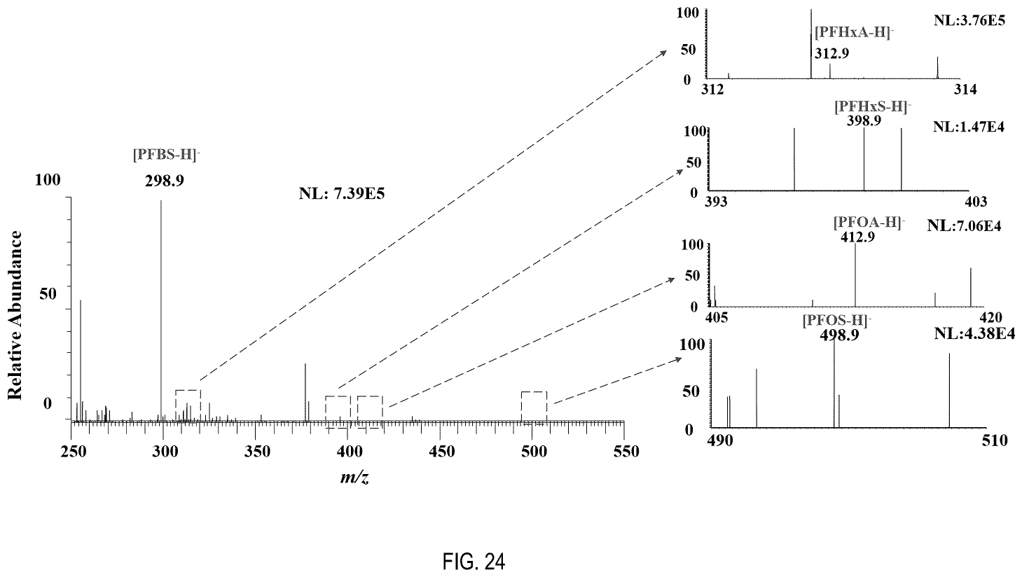

Wastewater from an anonymous location was directly applied to filter paper and the present PS-MS method using the optimized parameters outlined above was carried out. Different PFAS compounds, such as PFOA (measured m/z 412.96591, theoretical m/z 412.96643, mass error: −1.3 ppm), PFOS (measured m/z 498.92952, theoretical m/z 498.93022 and mass error: −1.4 ppm), PFHxA (measured m/z 312.97248, theoretical m/z 312.97281, mass error: −1.1 ppm), PFBS (measured m/z 298.94254, theoretical m/z 298.94299, mass error: −1.5 ppm) and perflurohexane sulfonic acid (PFHxS, measured m/z 398.93624, theoretical m/z 398.93660, mass error: −0.9 ppm) were observed, as illustrated in the acquired mass spectrum (FIG. 24). This result demonstrates the effectiveness and efficiency of the present PS-MS methods to analyze different water samples in a short period of time (about 2 min per sample).

### Example 9: Identification and Measurement of PFAS in Food Packaging Materials

Five commercially available food packaging materials were analyzed using the present PS-MS methods, and several different classes of PFAS were identified, such as per-fluorocarboxylate, per-fluorosulfonate and fluorotelomer unsaturated carboxylate. The primary purpose of using these PFAS in food packaging materials is to provide protection against grease, moisture, and heat, and to safeguard the food against external factors.

Each of the food packaging materials were cut into triangular shapes, to serve as paper spray emitters. To extract and ionize PFAS contained in the food packaging materials, MeOH (100%) was added directly onto the food packaging materials. This process was rapid and was completed in less than 1 min.

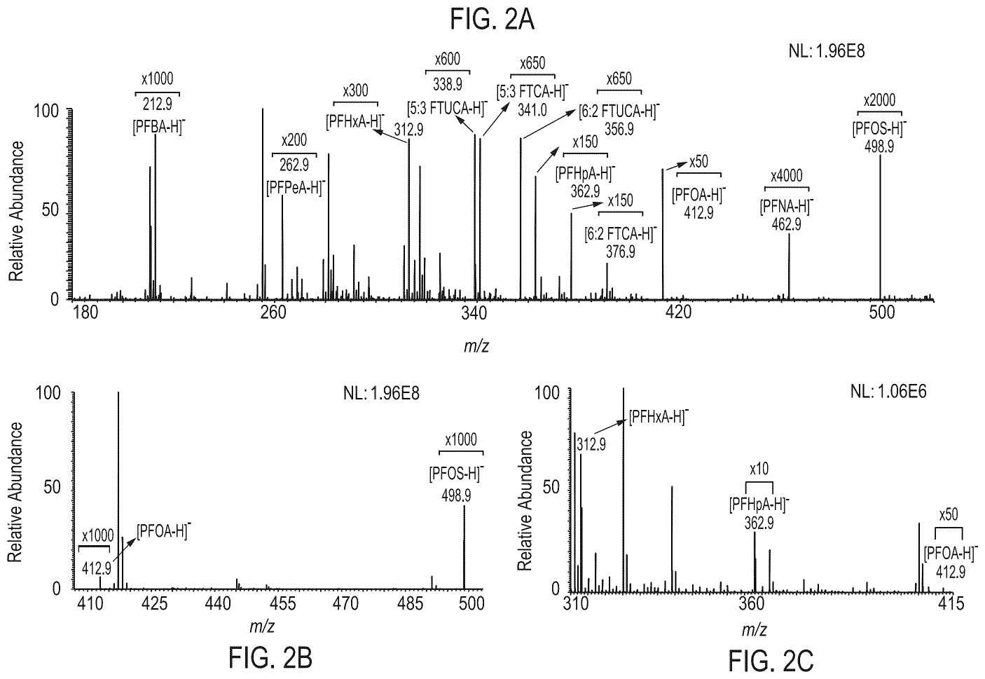

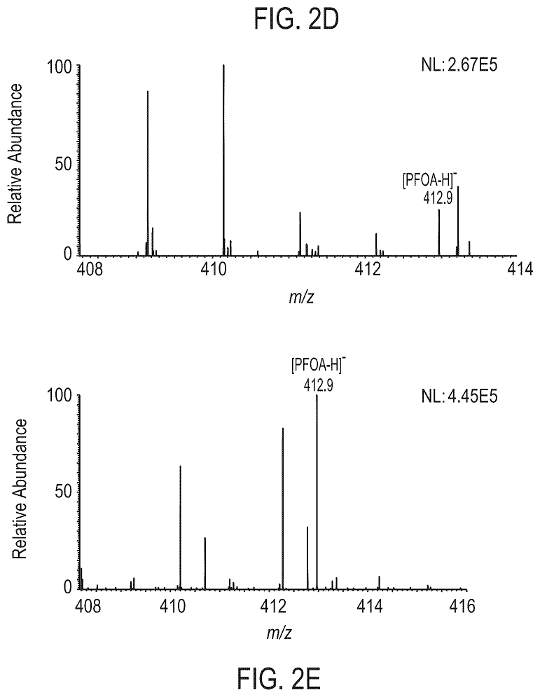

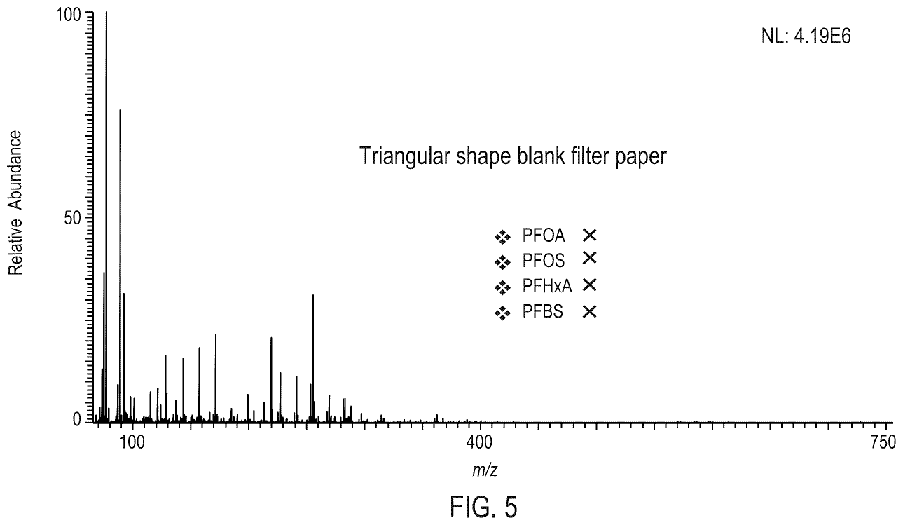

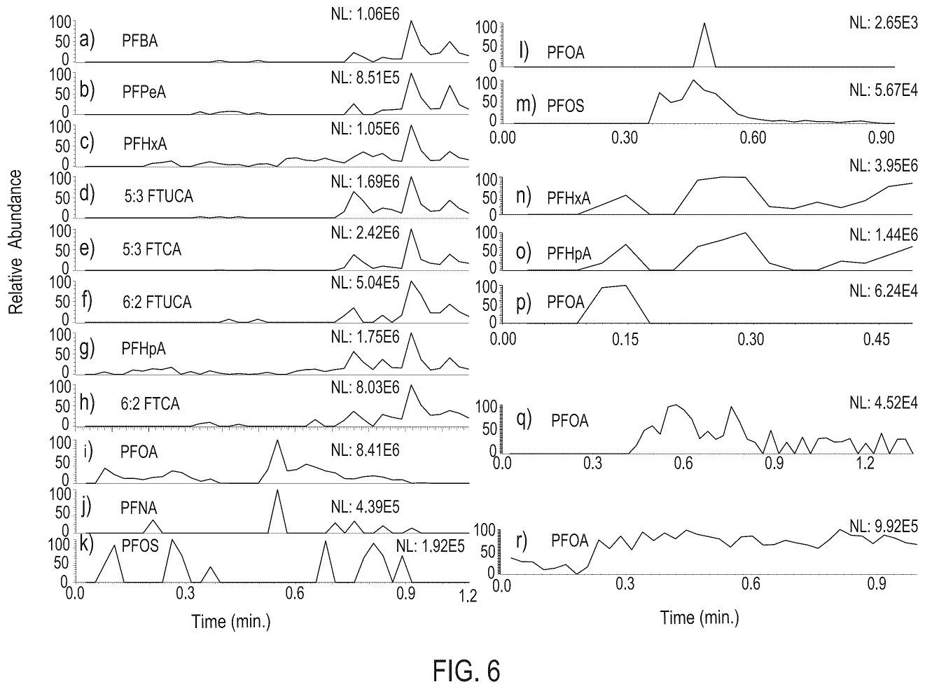

As illustrated in FIGS. 2A-E, the identified PFAS compounds included PFBA, PFPeA, PFHxA, 5:3 FTCA, 5:3 FTUCA, 6:2 FTUCA, PFHpA, 6:2 FTCA, PFOA, PFNA, and PFOS. This was confirmed by their accurate masses (mass error<5 ppm, FIG. 3 Table 2) and collision-induced dissociation (CID) analysis (see MS/MS data listed in FIG. 3 Table 2). The corresponding EIC spectra of these observed PFAS ions are illustrated in FIG. 6. Upon CID, the fragmentation of these PFAS ions occurs mainly via C—C or C—F cleavages or by losses of CO2 or SO3 (fragment ions are summarized in FIG. 3 Table 2), which is in agreement with ion dissociation behaviors of PFAS ions generated from standard PFAS samples (MS/MS results are summarized in Table 1, FIG. 39; MS spectra of PFAS ions from standard samples are also shown in FIG. 7).

Interestingly, all of the food packaging materials contained different types of PFAS. In particular, as illustrated in FIG. 2A, popcorn paper contained the most varieties of PFAS compounds. It was observed that PFOA is ubiquitous and was found in all of the tested samples. Results from the McDonald's wrapper (FIG. 2D) and Burger King French fry box (FIG. 2B) showed the existence of PFOA with reasonable intensity (1E4˜1E5), but the Burger King French fry box contained both classes of shorter chain PFAS (perfluoro carboxylate and perfluoro sulphonate). Furthermore, three shorter chain PFAS (PFHxA, PFHpA and PFOA) were identified from the Instant noodles box container (FIG. 2C). These experiments demonstrated the power of the present PS-MS methods in PFAS analysis, which is both sensitive and rapid, requiring no sample preparation (total time of about 1 min per sample).

### Example 10—Identification and Measurement of PFAS in Cosmetics

Three commercially available cosmetics products were examined to identify PFAS using the PS-MS methods described herein. Fisher brand qualitative p8 filter paper was first cut into a triangular shape and thoroughly cleaned using acetone, followed by a mixture of water and methanol, and finally methanol alone before use. A suitable amount of sample from the following cosmetic products was applied to provide a coating on at least a portion of the filter paper (e.g., where a suitable “coating” of mascara could result from pressing or dragging the mascara wand or applicator onto or along a portion of the filter paper, a suitable “coating” of concealer/foundation could result from pressing or swiping the concealer/foundation applicator or a swab dipped in the concealer/foundation across a portion of the filter paper, and a “coating” of eyeliner could be an applied as a line across a portion of the filter paper): Kiss Lengthen & Define Washable & Waterproof Mascara, Ruby Kisses Brush Concealer & Foundation, and Ruby Kisses Tattoo Felt Tip Eyeliner, which were all purchased from Amazon. The coated filter paper was positioned about 8 mm in front of the mass spectrometry inlet, a voltage of −3.5 kV was supplied via a high-voltage cable, and 100 μL of methanol was added to the cosmetic-coated filter paper for extraction, elution, and ionization.

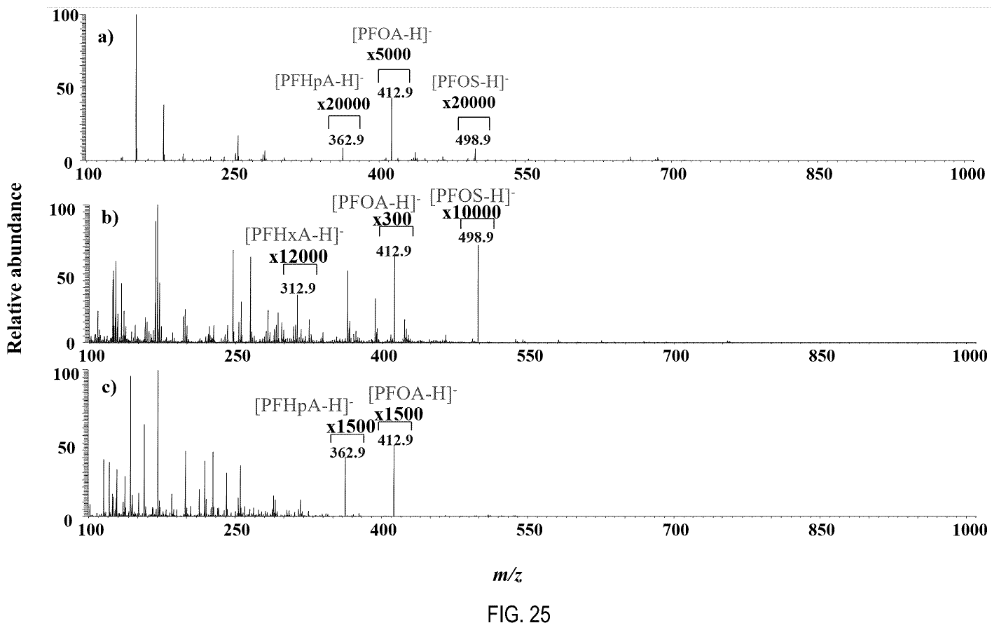

Using the present PS-MS methods, PS-MS analysis of the Kiss Lengthen & Define Washable & Waterproof Mascara detected and identified the presence of three different PFAS compounds within a mass error of ±5 ppm (FIG. 25A). The detected signal intensities were as follows: PFOS at 7.08E3 (experimental m/z 498.92960, theoretical m/z 498.93022, mass error: −1.2 ppm), PFOA at 1.46E5 (experimental m/z 412.96642, theoretical m/z 412.96643, mass error: −0.02 ppm), and PFHpA at 7.57E3 (experimental m/z 362.96963, theoretical m/z 362.96962, mass error: 0.03 ppm).

Using the present PS-MS methods, PS-MS analysis of the Ruby Kisses Brush Concealer & Foundation detected and identified the presence of three different PFAS compounds within a mass error of ±5 ppm (FIG. 25B). The detected signal intensities were PFOS at 3.04E3 (experimental m/z 498.92969, theoretical m/z 498.93022, mass error: −1.12 ppm), PFOA at 9.05E4 (experimental m/z 412.96591, theoretical m/z 412.96643, mass error: −1.3 ppm), and PFHxA at 1.24E3 (experimental m/z 312.97215, theoretical m/z 312.97281, mass error: −2.10 ppm).

Using the present PS-MS methods, PS-MS analysis of the Ruby Kisses Tattoo Felt Tip Eyeliner detected and identified the presence of two different PFAS compounds within a mass error of ±5 ppm (FIG. 25C). The signal intensities detected were PFOA at 1.28E3 (experimental m/z 412.96652, theoretical m/z 412.96643, mass error: 0.2 ppm) and PFHpA at 1.07E3 (experimental m/z 362.96988, theoretical m/z 362.96961, mass error: 0.7 ppm).

### Example 10—Detection of PFOA in Water Using Enhanced Sensitivity PS-MS

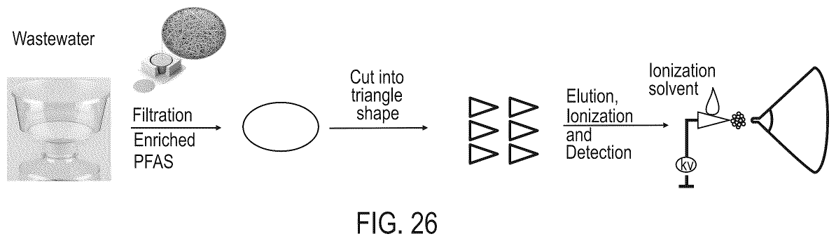

In this experiment, a 0.1 ppt PFOA in water sample was prepared. Two types of filter papers were used: a 47 mm P8 filter paper composed of cellulose fibers with a pore size of 20-25 μm, and a 47 mm cellulose ester membrane filter disc paper with a 0.2 μm pore size. 400 ml of water containing 0.1 ppt PFOA, was filtered using a vacuum filtration process, as illustrated in FIG. 26.

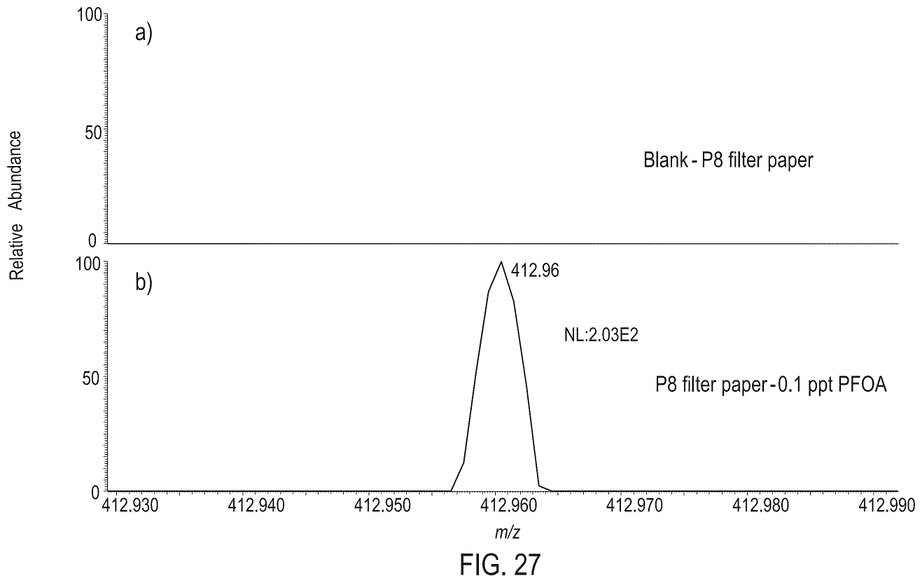

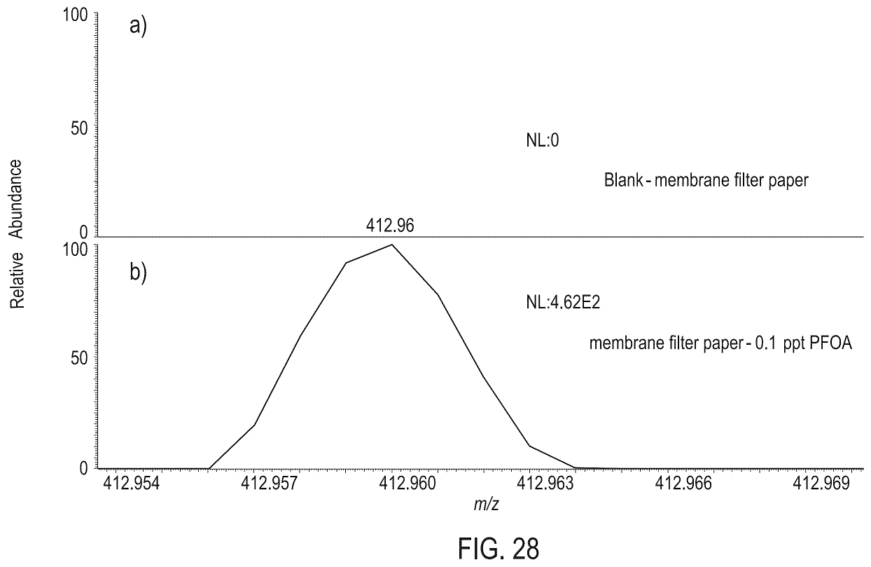

Following filtration, each of the filter papers were cut into triangular shapes, and attached to a high-voltage cable positioned about 8 mm in front of the mass spectrometry inlet. Methanol was employed for extraction, elution, and ionization. In a control sample, which contained no spiked PFOA, no PFAS compounds were detected. However, in the water samples containing 0.1 ppt PFOA, PFOA was successfully detected in both types of filter papers, as illustrated in FIGS. 27 and 28, with slight variations in intensity due to the differences in pore size or polarity between the two filter papers.

The present disclosure demonstrates the power of PS-MS and DPS-MS for trace analysis of PFAS in a variety of environmental samples. The present PS-MS and DPS-MS methods integrate extraction and ionization on the same filter paper for sample analysis with minimum sample preparation, thus enabling rapid detection of PFAS in various samples (e.g., about 3 min per sample or less). Remarkably, solid materials can be directly analyzed with the present PS-MS methods. For example, a solid material can be cut, torn, or otherwise manipulated into a desired sample shape or size (e.g. a food packaging material, a plant, a leaf, a vegetable, a fruit, a paper material, or a plastic material) and used in the present PS-MS method without the need for a filter paper.

The present DPS-MS methods integrate a desalting step together with elution and ionization, using the same filter paper to analyze samples with high background matrices. The present DPS-MS methods were demonstrated to be capable of detecting trace amounts of PFOA at 10 pM level in the presence of 50 mM of KCl and LODs for various PFAS is at low ppt level (with some embodiments providing as low as 1.2-4.5 ppt LOD). The high tolerance of the present DPS-MS methods to salt matrices allows the rapid analysis of PFAS in soil and sediment samples and extracts. In some embodiments, a small amount of soil (e.g., 40 mg) can be directly analyzed for PFAS using the present DPS-MS methods within 3 min without additional sample preparation.

These results clearly demonstrate that present PS-based MS techniques are fast, sensitive, and versatile for PFAS analysis in any material in which PFAS can be found (including, but not limited to, water, food packaging materials, and soil samples) which would find extensive real-world applications in the fields of environmental, food, and health sciences. PFAS are omnipresent in our environmental in trace levels and pose crucial environmental issues. These compounds are linked to health problems, including cancer, impaired fetus development, and suppressed vaccine response. Trace detection of these compounds from complex samples is crucial to secure ecosystems and human health. As such, the present methods would be beneficial in identifying effective mitigation strategies to PFAS.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure includes modifications and variations that are within the scope of the appended claims and their equivalents.

The following printed publications are incorporated herein by reference in their entireties, except for any definitions, subject matter disclaimers or disavowals, and except to the extent that the incorporated material is inconsistent with the express disclosure herein, in which case the language in this disclosure controls.

### REFERENCES

[symbol] ADDIN EN.REFLIST 1. Sivagami K, Sharma P, Karim A V, Mohanakrishna G, Karthika S, Divyapriya G, Saravanathamizhan R, Kumar A N. Electrochemical-based approaches for the treatment of forever chemicals: Removal of perfluoroalkyl and polyfluoroalkyl substances (PFAS) from wastewater. Science of The Total Environment. 2023; 861:160440.
2. Barreca S, Busetto M, Vitelli M, Colzani L, Clerici L, Dellavedova P. Online Solid-Phase Extraction LC-MS/MS: A Rapid and Valid Method for the Determination of Perfluorinated Compounds at Sub ng·L<sup>-1</sup>Level in Natural Water. Journal of Chemistry. 2018; 2018:3780825.
3. Stahl T, Mattern D, Brunn H. Toxicology of perfluorinated compounds. Environmental Sciences Europe. 2011; 23.
4. Glüge J, Scheringer M, Cousins I T, DeWitt J C, Goldenman G, Herzke D, Lohmann R, Ng C A, Trier X, Wang Z. An overview of the uses of per- and polyfluoroalkyl substances (PFAS). Environmental Science: Processes & Impacts. 2020; 22 (12):2345-2373.
5. Rodriguez K L, Hwang J-H, Esfahani A R, Sadmani A H M A, Lee W H. Recent Developments of PFAS-Detecting Sensors and Future Direction: A Review. Micromachines. 2020; 11 (7):667.
6. Brown H M, Fedick P W. Rapid, low-cost, and in-situ analysis of per- and polyfluoroalkyl substances in soils and sediments by ambient 3D-printed cone spray ionization mass spectrometry. Chemosphere. 2021; 272:129708.
7. Cheng J, Vecitis C D, Park H, Mader B T, Hoffmann M R. Sonochemical Degradation of Perfluorooctane Sulfonate (PFOS) and Perfluorooctanoate (PFOA) in Groundwater: Kinetic Effects of Matrix Inorganics. Environmental Science & Technology. 2010; 44 (1):445-450.
8. Zhuo Q, Deng S, Yang B, Huang J, Wang B, Zhang T, Yu G. Degradation of perfluorinated compounds on a boron-doped diamond electrode. Electrochimica Acta. 2012; 77:17-22.
9. Buck R C, Korzeniowski S H, Laganis E, Adamsky F. Identification and classification of commercially relevant per- and poly-fluoroalkyl substances (PFAS). Integr Environ Assess Manag. 2021; 17 (5):1045-1055.
10. Schymanski E L, Zhang J, Thiessen P A, Chirsir P, Kondic T, Bolton E E. Per- and Polyfluoroalkyl Substances (PFAS) in PubChem: 7 Million and Growing. Environmental Science & Technology. 2023; 57 (44):16918-16928.
11. Vierke L, Staude C, Biegel-Engler A, Drost W, Schulte C. Perfluorooctanoic acid (PFOA)—main concerns and regulatory developments in Europe from an environmental point of view. Environmental Sciences Europe. 2012; 24 (1):16.
12. Leung S C E, Shukla P, Chen D, Eftekhari E, An H, Zare F, Ghasemi N, Zhang D, Nguyen N-T, Li Q. Emerging technologies for PFOS/PFOA degradation and removal: A review. Science of The Total Environment. 2022; 827:153669.
13. Seró R, Ayala-Cabrera J F, Santos F J, Moyano E. Paper spray-atmospheric pressure photoionization-high resolution mass spectrometry for the direct analysis of neutral fluorinated compounds in waterproof impregnation sprays. Anal Chim Acta. 2022; 1204:339720.
14. Phung T V, Nguyen T N, Phan Thi L-A, Pham H V, Duong H A. Determination of Per- and Polyfluoroalkyl Substances in Craft Villages and Industrial Environments of Vietnam. Journal of Analytical Methods in Chemistry. 2021; 2021:5564994.
15. Ramírez Carnero A, Lestido-Cardama A, Vazquez Loureiro P, Barbosa-Pereira L, Rodríguez Bernaldo de Quirós A, Sendón R. Presence of Perfluoroalkyl and Polyfluoroalkyl Substances (PFAS) in Food Contact Materials (FCM) and Its Migration to Food. Foods. 2021; 10 (7).
16. Kurwadkar S, Dane J, Kanel S R, Nadagouda M N, Cawdrey R W, Ambade B, Struckhoff G C, Wilkin R. Per- and polyfluoroalkyl substances in water and wastewater: A critical review of their global occurrence and distribution. Sci Total Environ. 2022; 809:151003.
17. Brusseau M L, Anderson R H, Guo B. PFAS concentrations in soils: Background levels versus contaminated sites. Sci Total Environ. 2020; 740:140017.
18. Moody C A, Martin J W, Kwan W C, Muir D C, Mabury S A. Monitoring perfluorinated surfactants in biota and surface water samples following an accidental release of fire-fighting foam into Etobicoke Creek. Environ Sci Technol. 2002; 36 (4):545-551.
19. Urtiaga A, Fernández-González C, Gómez-Lavín S, Ortiz I. Kinetics of the electrochemical mineralization of perfluorooctanoic acid on ultrananocrystalline boron doped conductive diamond electrodes. Chemosphere. 2015; 129:20-26.
20. Wu C, Wang Q, Chen H, Li M. Rapid quantitative analysis and suspect screening of per- and polyfluorinated alkyl substances (PFASs) in aqueous film-forming foams (AFFFs) and municipal wastewater samples by Nano-ESI-HRMS. Water Research. 2022; 219:118542.
21. Marsh K, Bugusu B. Food Packaging—Roles, Materials, and Environmental Issues. Journal of Food Science. 2007; 72 (3):R 39-R 55.
22. Lau O W, Wong S K. Contamination in food from packaging material. J Chromatogr A. 2000; 882 (1-2):255-270.
23. Gallart-Ayala H, Núñez O, Lucci P. Recent advances in LC-MS analysis of food-packaging contaminants. Trends in Analytical Chemistry. 2013; 42:99-124.
24. Richardson S D. Environmental Mass Spectrometry: Emerging Contaminants and Current Issues. Analytical Chemistry. 2012; 84 (2):747-778.
25. Nakayama S F, Yoshikane M, Onoda Y, Nishihama Y, Iwai-Shimada M, Takagi M, Kobayashi Y, Isobe T. Worldwide trends in tracing poly- and perfluoroalkyl substances (PFAS) in the environment. TrAC Trends in Analytical Chemistry. 2019; 121:115410.
26. Point A D, Holsen T M, Fernando S, Hopke P K, Crimmins B S. Towards the development of a standardized method for extraction and analysis of PFAS in biological tissues. Environmental Science: Water Research & Technology. 2019; 5 (11):1876-1886.
27. Ahmadireskety A, Da Silva B F, Townsend T G, Yost R A, Solo-Gabriele H M, Bowden J A. Evaluation of extraction workflows for quantitative analysis of per- and polyfluoroalkyl substances: A case study using soil adjacent to a landfill. Science of The Total Environment. 2021; 760:143944.
28. Kourtchev I, Hellebust S, Heffernan E, Wenger J, Towers S, Diapouli E, Eleftheriadis K. A new on-line SPE LC-HRMS method for the analysis of Perfluoroalkyl and Polyfluoroalkyl Substances (PFAS) in PM2.5 and its application for screening atmospheric particulates from Dublin and Enniscorthy, Ireland. Science of The Total Environment. 2022; 835:155496.
29. Al Amin M, Sobhani Z, Liu Y, Dharmaraja R, Chadalavada S, Naidu R, Chalker J M, Fang C. Recent advances in the analysis of per- and polyfluoroalkyl substances (PFAS)—A review. Environmental Technology & Innovation. 2020; 19:100879.
30. Honda M, Robinson M, Kannan K. A rapid method for the analysis of perfluorinated alkyl substances in serum by hybrid solid-phase extraction. Environmental Chemistry. 2018; 15:92-99.
31. Simon F, Gehrenkemper L, von der Au M, Wittwer P, Roesch P, Pfeifer J, Cossmer A, Meermann B. A fast and simple PFAS extraction method utilizing HR-CS-GFMAS for soil samples. Chemosphere. 2022; 295:133922.
32. Zabaleta I, Negreira N, Bizkarguenaga E, Prieto A, Covaci A, Zuloaga O. Screening and identification of per- and polyfluoroalkyl substances in microwave popcorn bags. Food Chemistry. 2017; 230:497-506.
33. Yeung L W Y, De Silva A O, Loi E I H, Marvin C H, Taniyasu S, Yamashita N, Mabury S A, Muir D C G, Lam P K S. Perfluoroalkyl substances and extractable organic fluorine in surface sediments and cores from Lake Ontario. Environment International. 2013; 59:389-397.
34. Rankin K, Mabury S A, Jenkins T M, Washington J W. A North American and global survey of perfluoroalkyl substances in surface soils: Distribution patterns and mode of occurrence. Chemosphere. 2016; 161:333-341.
35. Schroeder T, Bond D, Foley J. PFAS soil and groundwater contamination via industrial airborne emission and land deposition in SW Vermont and Eastern New York State, USA. Environmental Science: Processes & Impacts. 2021; 23 (2):291-301.
36. Liu J, Wang H, Manicke N E, Lin J-M, Cooks R G, Ouyang Z. Development, Characterization, and Application of Paper Spray Ionization. Analytical Chemistry. 2010; 82 (6):2463-2471.
37. Wang Q, Bhattarai M, Zhao P, Alnsour T, Held M, Faik A, Chen H. Fast and Sensitive Detection of Oligosaccharides Using Desalting Paper Spray Mass Spectrometry (DPS-MS). J Am Soc Mass Spectrom. 2020; 31 (10):2226-2235.
38. Chiu K-Y, Wang Q, Gunawardena H P, Held M, Faik A, Chen H. Desalting paper spray mass spectrometry (DPS-MS) for rapid detection of glycans and glycoconjugates. International Journal of Mass Spectrometry. 2021; 469:116688.
39. Wang H, Liu J, Cooks R G, Ouyang Z. Paper spray for direct analysis of complex mixtures using mass spectrometry. Angew Chem Int Ed Engl. 2010; 49 (5):877-880.
40. Liu J, Wang H, Manicke N E, Lin J M, Cooks R G, Ouyang Z. Development, characterization, and application of paper spray ionization. Anal Chem. 2010; 82 (6):2463-2471.
41. Narayanan R, Song X, Chen H, Zare R N. Teflon Spray Ionization Mass Spectrometry. J Am Soc Mass Spectrom. 2020; 31 (2):234-239.
42. Zhang Y, Ju Y, Huang C, Wysocki V H. Paper Spray Ionization of Noncovalent Protein Complexes. Analytical Chemistry. 2014; 86 (3):1342-1346.
43. Song X, Chen H, Zare R N. Conductive Polymer Spray Ionization Mass Spectrometry for Biofluid Analysis. Analytical Chemistry. 2018; 90 (21):12878-12885.
44. Banerjee S, Basheer C, Zare R N. A Study of Heterogeneous Catalysis by Nanoparticle-Embedded Paper-Spray Ionization Mass Spectrometry. Angew Chem Int Ed Engl. 2016; 55 (41):12807-12811.
45. Moura A C M, Lago I N, Cardoso C F, Dos Reis Nascimento A, Pereira I, Vaz B G. Rapid monitoring of pesticides in tomatoes (Solanum lycopersicum L.) during pre-harvest intervals by paper spray ionization mass spectrometry. Food Chem. 2020; 310:125938.
46. Tosato F, Correia R M, Oliveira B G, Fontes A M, França H S, Coltro W K T, Filgueiras P R, Romão W. Paper spray ionization mass spectrometry allied to chemometric tools for quantification of whisky adulteration with additions of sugarcane spirit. Analytical Methods. 2018; 10 (17):1952-1960.
47. Poole J J, Gómez-Ríos G A, Boyaci E, Reyes-Garcés N, Pawliszyn J. Rapid and Concomitant Analysis of Pharmaceuticals in Treated Wastewater by Coated Blade Spray Mass Spectrometry. Environ Sci Technol. 2017; 51 (21):12566-12572.
48. Shoemaker J, Tettenhorst D. Method 537.1: determination of selected per- and polyfluorinated alkyl substances in drinking water by solid phase extraction and liquid chromatography/tandem mass spectrometry (LC/MS/MS). National Center for Environmental Assessment, Washington, DC. 2018.
49. Zhang Z, Cooks R G, Ouyang Z. Paper spray: a simple and efficient means of analysis of different contaminants in foodstuffs. Analyst. 2012; 137 (11):2556-2558.
50. Su Y, Wang H, Liu J, Wei P, Cooks R G, Ouyang Z. Quantitative paper spray mass spectrometry analysis of drugs of abuse. Analyst. 2013; 138 (16):4443-4447.
51. Chiu K Y, Wang Q, Gunawardena H P, Held M, Faik A, Chen H. Desalting Paper Spay Mass Spectrometry (DPS-MS) for Rapid Detection of Glycans and Glycoconjugates. Int J Mass Spectrom. 2021; 469.
52. Liu J, Wang H, Cooks R G, Ouyang Z. Leaf Spray: Direct Chemical Analysis of Plant Material and Living Plants by Mass Spectrometry. Analytical Chemistry. 2011; 83 (20):7608-7613.
53. Huang Z, Zhang X, Wang X, Deji Z, Lee H K. Occurrence of Perfluoroalkyl and Polyfluoroalkyl Substances in Ice Cream, Instant Noodles, and Bubble Tea. Journal of Agricultural and Food Chemistry. 2022; 70 (35):10836-10846.
54. Li J, Li X, Da Y, Yu J, Long B, Zhang P, Bakker C, McCarl B A, Yuan J S, Dai S Y. Sustainable environmental remediation via biomimetic multifunctional lignocellulosic nano-framework. Nat Commun. 2022; 13 (1):4368.

What is claimed is:

1. A method for detecting the presence of one or more per- and polyfluorinated alkyl substances (P FAS) in a sample, the method comprising:
analyzing the sample using a paper spray mass spectrometry (PS-MS) technique to detect the one or more PFAS, wherein the PS-MS technique uses high voltage spray ionization.

2. The method of claim 1, wherein the PS-MS technique comprises:
applying the sample to a filter paper;
positioning the filter paper in front of a mass spectrometry inlet;
eluting the one or more PFAS by wetting the filter paper with an elution solvent; and
applying a voltage to the wetted paper to ionize the one or more PFAS.

3. The method of claim 1, wherein the method provides detection of the one or more PFAS within a limit of detection of about 0.01 ppt to about 100 ppt.

4. The method of claim 1, wherein the method is completed within about 3 minutes.

5. The method of claim 1, wherein the sample is selected from water, soil, air, plants, food, sludge, vegetables, meats, packaging materials, plastic products, toys, cosmetics, agricultural products, pharmaceutical products, electronics, consumer products, and solvent extracts thereof.

6. The method of claim 1, wherein the sample is an unprocessed sample.

7. The method of claim 1, wherein prior to analyzing the sample, the method further comprises extraction of the sample with a solvent, wherein analyzing the sample comprises analyzing the sample extract, and wherein the method is completed within about 6 minutes.

8. The method of claim 1, wherein the sample is a solid material containing one or more PFAS, wherein analyzing the sample using a PS-MS comprises directly analyzing the solid material, and wherein applying the PS-MS technique comprises:
cutting the solid material containing one or more PFAS into a cut sample shape;
positioning the cut sample shape in front of a mass spectrometry inlet;
eluting the one or more PFAS by wetting the cut sample shape with an elution solvent; and
applying a voltage to the wetted cut sample shape to ionize the one or more PFAS.

9. The method of claim 8, wherein the solid material is a food packaging material, a plant, a leaf, a vegetable, a fruit, a paper material, or a plastic material.

10. The method of claim 2, wherein about 1 μL to about 100 μL of sample is applied to the filter paper at least one time and up to four times, wherein positioning the filter paper in front of the mass spectrometry inlet comprises positioning the filter paper about 2 mm to about 50 mm in front of the mass spectrometry inlet, wherein the voltage ranges from about 1 kV to about 10 kV, and wherein the elution solvent is an organic solvent, water, or a combination of organic solvent and water, or a solvent containing a derivatizing reagent.

11. The method of claim 8, wherein positioning the cut sample shape in front of the mass spectrometry inlet comprises positioning the cut sample shape about 2 mm to about 50 mm in front of the mass spectrometry inlet, wherein the voltage ranges from about 1 kV to about 10 kV, and wherein the elution solvent is an organic solvent, water, or a combination of organic solvent and water, or a solvent containing a derivatizing reagent.

12. A method for detecting the presence of one or more per- and polyfluorinated alkyl substances (PFAS) in a sample, the method comprising:
obtaining a sample; and
analyzing the sample using a paper spray mass spectrometry (PS-MS) technique to detect the one or more PFAS,
wherein the sample analyzed by the PS-MS technique is either unprocessed or minimally processed.

13. The method of claim 12, wherein the PS-MS technique comprises:
applying the sample to a filter paper;
positioning the filter paper in front of a mass spectrometry inlet;
eluting the one or more PFAS by wetting the filter paper with an elution solvent; and
applying a voltage to the wetted paper to ionize the one or more PFAS.

14. The method of claim 12, wherein the method provides detection of the one or more PFAS within a limit of detection of about 0.01 ppt to about 100 ppt.

15. The method of claim 12, wherein the method is completed within about 3 minutes.

16. The method of claim 12, wherein the sample is selected from water, soil, air, plants, food, sludge, vegetables, meats, packaging materials, plastic products, toys, cosmetics, agricultural products, pharmaceutical products, electronics, consumer products, and solvent extracts thereof.

17. The method of claim 12, wherein prior to analyzing the sample, the method further comprises extraction of the sample with a solvent to provide a sample extract, wherein analyzing the sample comprises analyzing the sample extract, and wherein the method is completed within about 6 minutes.

18. A method for detecting the presence of one or more per- and polyfluorinated alkyl substances (P FAS) in a sample containing one or more ion suppression matrices, the method comprising:
obtaining the sample; and
analyzing the sample using a desalting paper spray mass spectrometry (DPS-MS) technique to detect the one or more PFAS, the DPS-MS technique comprising:
applying the sample to a filter paper;
performing a desalting step to remove the ion suppression matrices from the filter paper;
positioning the filter paper in front of a mass spectrometry inlet;
eluting the one or more PFAS by wetting the filter paper with an elution solvent; and
applying a voltage to the wetted filter paper to ionize the one or more PFAS.

19. The method of claim 18, wherein the desalting step comprises, after applying the prepared sample to the filter paper, adding water to the filter paper to wick away the ion suppression matrices.

20. The method of claim 18, wherein the sample is an unprocessed or minimally processed sample.
